(19) United States
(12) Patent Application Publication
(10) Pub. No.: US 2021/0405057 A1
(43) Pub. Date: Dec. 30, 2021

(54) METHODS AND COMPOSITIONS FOR DETERMINING THE COMPOSITION OF A TUMOR MICROENVIRONMENT

(71) Applicant: Pierian Biosciences, LLC, Franklin, TN (US)

(72) Inventors: Norman Purvis, Franklin, TN (US); Matthew Westfall, Franklin, TN (US)

(21) Appl. No.: 17/292,269

(22) PCT Filed: Nov. 8, 2019
(86) PCT No.: PCT/US2019/060521

§ 371 Date: May 7, 2021

Related U.S. Application Data
(60) Provisional application No. 62/758,393, filed on Nov. 9, 2018.

(51) Int. Cl. G01N 33/574; G01N 33/50

(52) U.S. Cl. CPC G01N 33/57492; G01N 33/5011

(57) ABSTRACT

The disclosure relates to the field of immuno-oncology. More specifically, the disclosure relates to methods and compositions for performing single-cell phenotypic and functional analysis of immune and cancer cells disposed within a tumor microenvironment. The methods and compositions permit the determination of whether a subject with a solid tumor is likely to respond to a particular immunomodulator, and also permit the treatment of a solid tumor in a subject by facilitating the selection of an immunomodulator suitable for treating the solid tumor in the subject.

### CROSS-REFERENCE TO RELATED APPLICATIONS

This application claims the benefit of and priority to U.S. Provisional Patent Application No. 62/758,393, filed Nov. 9, 2018, the entire disclosure of which is incorporated by reference herein.

### FIELD OF THE INVENTION

The disclosure relates to the field of immuno-oncology. More specifically, the disclosure relates to methods and compositions for dissociating and analyzing tumor samples to determine the phenotypic composition of the tumor microenvironment, evaluate the functional status of the infiltrating immune cells, quantify the expression of immune modulatory receptors and ligands, and predict response to immunotherapy.

### BACKGROUND

The discovery of checkpoint inhibitors in cancer treatment has provided new therapies directed to treating cancers that, until this discovery, have evaded successful treatment because the cancers have evolved mechanisms for evading a subject's immune system. However, despite the significant advances made to date in treating various cancers with checkpoint inhibitors, the currently available biomarker and diagnostics are poor predictors of response and thus significant numbers of patients remain unresponsive to immunotherapy treatment. Furthermore, solid tumors may be harder to evaluate for potential treatment options, given the complexity of the tumor microenvironment (TME) of a solid tumor, which contains epithelial cells, endothelial cells, mesenchymal cells, stromal cells, cancer cells and immune cells. In certain solid tumors, the cancer cells influence of the activity or inactivity of the immune cells in the TME.

Accordingly, despite the advances made to date, there is still a need for methods and compositions that can determine the composition of the TME of a solid tumor, which can then be used to determine what immunomodulators, either alone or in combination with other cancer drugs, may be effective in treating a tumor in a given subject.

### SUMMARY OF THE INVENTION

The present invention is based, in part, upon the discovery that it is possible to determine the composition of a solid tumor microenvironment TME and to perform single-cell phenotypic and functional analysis of immune and cancer cells disposed within a TME. More specifically, the methods and compositions described herein facilitate the quantitative characterization of immunosuppressive phenotypes as well as the cellular expression of targetable immune modulatory (e.g., checkpoint) receptors and their ligands within a TME. The methods and compositions can be used to determine whether a subject with a solid tumor is likely to respond to a particular immunomodulator. The methods and compositions also facilitate the treatment of a solid tumor in a given subject by aiding the selection of an immunomodulator suitable for treating the solid tumor in the subject.

Furthermore, the methods and compositions of the present invention provide a drug discovery/evaluation tool for (a) early stage immunotherapeutic exploratory studies; (b) providing mechanism-based proof of principal in pre-clinical studies, (c) patient stratification in clinical trials; and (d) selecting an immunomodulator most likely to be effective in treating a cancer in a subject.

The methods described herein also facilitate a phenotypic analysis of tumor-infiltrating leukocyte (TILs) thereby providing accurate and highly reproducible data sets under validated conditions. Accordingly, the breadth and quality of data collected according to the methods described herein provides advantages over conventional cellular evaluation approaches, for example, conventional immuno-histochemical approaches.

In one aspect, the disclosure relates to a method of determining the composition of a solid tumor microenvironment. The method includes combining a single cell suspension of cells derived from a solid tumor with a plurality of labeling agents capable of binding a corresponding plurality of cell surface markers expressed on cancer cells and/or immune cells, wherein the cell surface markers comprise cell-type markers, immune modulatory receptors (IMRs) and IMR-ligand markers (IMR-Ls), and permitting the agents to simultaneously hind to cancer cells, immune cells or both cancer cells and immune cells present in the single cell suspension to produce labeled cells. The method further includes determining the presence and/or amount of the labeled cells by cytometry thereby to (i) determine the presence and/or amount of cancer cells, immune cells, or both cancer cells and immune cells present in the microenvironment of the solid tumor and (ii) determine whether the cancer cells, immune cells or both the cancer cells and immune cells express at least one of the IMRs and/or at least one of the IMR-Ls, thereby to determine the solid tumor microenvironment.

In another aspect, the disclosure relates to a method of determining whether a subject with solid tumor is likely to respond to an immunomodulator. The method includes combining a single cell suspension of cells derived from a solid tumor with a plurality of labeling agents capable of binding a corresponding plurality of cell surface markers expressed on cancer cells and/or immune cells, wherein the cell surface markers comprise cell-type markers, immune modulatory receptors (IMRs) and IMR-ligands (IMR-Ls), and permitting the agents to simultaneously bind to cancer cells, immune cells, or both cancer cells an immune cells present in the single cell suspension to produce labeled cells. The method further includes combining at least a portion of the single cell suspension of cells with an immunomodulator and determining (i) the presence and/or amount of the labeled cells by cytometry thereby to determine the presence and/or amount of cancer cells, immune cells or both cancer cells and immune cells present in the solid tumor and whether cancer cells, immune cells or both the cancer cells and immune cells express at least one of the IMRs and/or at least one of the IMR-Ls and (ii) the effect of the immunomodulator on the cellular markers on or in the cancer cells, immune cells or both the cancer cells and immune cells, thereby to determine whether the subject is likely to respond to the immunomodulator.

In another aspect, the disclosure relates to a method of treating a solid tumor in a subject in need thereof. The method includes administering an effective amount of an immunomodulator to the subject thereby to treat the solid tumor. The immunomodulator is selected by a method that includes combining a single cell suspension of cells derived from the solid tumor with a plurality of labeling agents capable of binding a corresponding plurality of cell surface markers expressed on cancer cells and/or immune cells, wherein the cell surface markers comprise cell-type markers, immune modulatory receptors (IMRs) and IMR-ligands (IMR-Ls), and permitting the agents to simultaneously bind to the cancer cells, immune cells or both cancer and immune cells present in the single cell suspension to produce labeled cells. The method further includes combining at least a portion of the single cell suspension of cells with an immunomodulator and determining (i) the presence and/or amount of the labeled cells by cytometry thereby to determine the presence and/or amount of cancer cells, immune cells, or both cancer cells and immune cells present in the solid tumor and whether the cancer cells, immune cells or both the cancer cells and immune cells express at least one of the IMRs and/or at least one of the IMR-Ls and (ii) the effect of the immunomodulator on the cellular markers on or in the cancer cells, immune cells or both the cancer cells and immune cells thereby to determine whether the subject is likely to respond to the immunomodulator.

In certain embodiments of the above aspects, the cytometry is flow cytometry, mass cytometry, image cytometry, and/or a single cell technology (SCT).

In certain embodiments of the above aspects, the labeling agents are selected from the group consisting of oligonucleotides, fluorophores, infrared labels, and heavy metal labels.

In certain embodiments of the above aspects, wherein the immune cells comprise lymphocytes (e.g., T cells (e.g., CD4+ T cells, CD8+ T cells, Tregs), B-cells, and natural killer cells), myeloid cells (e.g., dendritic cells, macrophages, and myeloid-derived suppressor cells), or a combination thereof.

In certain embodiments, the cell surface markers further comprise cell activation markers.

In certain embodiments, the cell-type markers comprise markers expressed on lymphocytes (e.g., T cells (e.g., CD4+ T cells, CD8+ T cells, Tregs), B-cells, and natural killer cells), and/or myeloid cells (e.g., dendritic cells, macrophages, and myeloid-derived suppressor cells). In certain embodiments, the cell-type markers are cancer cell markers, including CD44, CD47, CD49f, CD271, CD326, cytokeratin (intracellular), E-cadherin, and/or vimentin.

In certain embodiments, the cell activation markers comprise CD25, CD26, CD27, CD28, CD38, CD40, CD44, CD62L, CD69, CD80, CD86, CD95, CD95L, CD127, CCR7 (CD197), and/or functional markers, e.g., IFNγ, TNFα, and/or other cytokines and/or Granzyme B.

In certain embodiments, the IMR or IMR-L markers comprise PD-1 (CD279), PD-L1 (CD274), CTLA-4 (CD152), LAGS (CD223), OX40 (CD134), TIM3 (CD366), GITR (CD357), 4-1BB (CD137), KIR (CD158B), 2B4 (CD244), ICOS (CD278), IDO, TIGIT, CD73, CD39, CD172a (SIRPa), B7H4 (B7S1), VISTA (B7-H5), CD355 (CRTAM), KLRG1, CD160 (BY55, NK1, NK28), CD30 (TNFRSF8), CD224 (GGT1), CD226, CD272 (BTLA), and/or CD115 (CSF-1R).

In certain embodiments, the method further incudes the step of combining the cells with an immunomodulator. In certain embodiments, the method further includes determining the effect of the immunomodulator on the expression of at least a portion of the cellular markers expression on the cancer and/or immune cells. The immunomodulator can be combined with the single cell suspension before, during or after the step of combining a single cell suspension of cells derived from a solid tumor with a plurality of labeling agents capable of binding a corresponding plurality of cell surface markers expressed on cancer cells and/or immune cells.

In certain embodiments, the plurality of different labeled cells are detected at the same time during cytometry. In certain embodiments, the plurality of different cell surface markers are detected at the same time during cytometry. In certain embodiments, at least 14 different cell surface markers are detected at the same time.

In certain embodiments, receptor-ligand interactions between the labeled cells can be detected and optionally quantified. In certain embodiments, receptor-ligand interactions comprise interactions between a checkpoint inhibitor and its cognate ligand. In certain embodiments, the receptor-ligand interactions can be selected from the interactions between PD-1 and PD-L1, CTLA-4 and B7-1 and/or B7-2, TIM-3 and Gal9, GITR and GITRL, OX-40 and OX40L, CD-27 and CD70, 4-1BB and 4-1BBL, and/or CD-40L and CD40.

In certain embodiments, the presence and/or amount of the cell activation markers, IMR markers, IMR-L markers or a combination of the activation markers and the IMR and/or IMR-L markers expressed on the cancer cells and/or immune cells is determined.

The description above describes multiple aspects and embodiments of the invention. The patent application specifically contemplates all combinations and permutations of the aspects and embodiments. These and other aspects and features of the invention are described in the following detailed description and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings, in which:

FIG. 1 depicts a gating tree for automated metrics extraction from pre-determined gating routines for the identification of Tregs and expression of Granzyme B or cytokines in a Treg staining panel. Flow cytometric data was analyzed utilizing a combination of the Qognit software package Ryvett and Verity Software House software package Winlist. The gating tree represents the Boolean logic that was used to identify cell populations, capturing all cell populations and segregating them into more discrete populations as the gating tree branches.

FIG. 2 depicts a gating tree for automated metrics extraction from pre-determined gating routines for the identification of T cells, B cells, NK cells, and monocyte/macrophage cells and the expression of IMRs and IMR-Ls on identified cell subsets. Flow cytometric data was analyzed utilizing a combination of the Qognit software package Ryvett and Verity Software House software package Winlist. The gating tree represents the Boolean logic that was used to identify cell populations, capturing all cell populations and segregating them into more discrete populations as the gating tree branches.

FIG. 3 depicts immune modulatory receptor (IMR)/immunomodulatory ligand (IMR-L) pairs detectable according to the methods of the present invention.

FIG. 4A depicts surface and intracellular phenotypic flow cytometry plots for a dissociated tumor sample showing the presence of CD3+, CD4+, CD8+ and CD4+CD25^{hi}FoxP3+ Treg cells. FIG. 4B depicts the associated numerical plotting (represented as percent positive) plots showing the prevalence of CD3+, CD4+, CD8+ and CD4+CD25^{hi}FoxP3+ Treg cells. Elevated levels of CD4+CD25^{hi}FoxP3+ Tregs suggests (a) an immunosuppressive signature with tumor spreading and (b) poor prognosis and objective response. (RD-PBMC=PBMC reference donor used across all assays.)

FIG. 5 depicts flow cytometry histogram plots for one or more basal or induced intracellular detectable antigens and the associated numerical plotting (represented as percent positive) of the gated histogram. Intracellular cytokine expression (IFNγ and TNFα) was determined in CD4+ and CD8+ T cells isolated from the tumor microenvironment of breast, lung, and renal tumors in the presence or absence of Leukocyte Activation Cocktail with BD GolgiPlug™. Triangles represent breast tumors, squares represent lung tumors, diamonds represent renal tumors, and circles represent data from a PBMC reference donor (RD-PBMC).

FIG. 6 depicts flow cytometry histogram plots and the associated numerical plotting (represented as percent positive) of the gated histogram showing expression of Granzyme B CD8+ cytotoxic T cells of breast, lung, and renal tumors as compared to a control (FMO) and a healthy donor PBMC. Triangles represent breast tumors, squares represent lung tumors, diamonds represent renal tumors, and circles represent data from a PBMC reference donor. MFI=mean fluorescence intensity.

FIG. 7 depicts surface phenotypic flow cytometry plots for a dissociated tumor sample showing the presence of CD45+ leukocytes, CD326+ epithelial tumor cells, CD14+ monocyte/macrophages and CD4+ and CD8+ T cells.

FIG. 8 depicts flow cytometry histogram plots showing the expression of the immune inhibitory checkpoint TIGIT (T cell immunoreceptor with Ig and ITIM domains) expression on CD4+, CD8+, and CD14+ cells but no expression on the CD326+ epithelial tumor cells. FMO=control.

FIG. 9 depicts numerical representation (expressed as a percentage) of several IMRs or IMR-Ls expressed on CD326+ epithelial tumor cells obtained from a cohort of dissociated breast, lung, and renal tumors. Medium-grey squares represent expression levels in breast tumors, light-grey squares represent expression levels in lung tumors, and dark-grey squares represent expression levels in renal tumors.

FIG. 10 depicts numerical representation (expressed as a percentage) of several IMRs or IMR-Ls expressed on CD14+ myeloid cells obtained from a cohort of dissociated breast, lung, and renal tumors. Medium-grey squares represent expression levels in breast tumors, light-grey squares represent expression levels in lung tumors, and dark-grey squares represent expression levels in renal tumors.

FIG. 11 depicts numerical representation (expressed as a percentage) of several IMRs or IMR-Ls expressed on CD8+ T cells obtained from a cohort of dissociated breast, lung, and renal tumors. Medium-grey squares represent expression levels in breast tumors, light-grey squares represent expression levels in lung tumors, and dark-grey squares represent expression levels in renal tumors.

### DETAILED DESCRIPTION

The present invention is based, in part, upon the discovery that it is possible to determine the composition of a solid tumor microenvironment and to perform single-cell phenotypic and functional analysis of immune and cancer cells disposed within a tumor microenvironment (TME). More specifically, the methods and compositions described herein facilitate the quantitative characterization of immunosuppressive phenotypes as well as the cellular expression of targetable immune modulatory (e.g., checkpoint) receptors and their ligands within a TME. The methods and compositions can be used to determine whether a given subject with a solid tumor is likely to respond to treatment with a particular immunomodulator so that the subject is not exposed to agents unlikely to provide a positive treatment outcome but rather are treated with one of more agents selected to achieve a positive treatment outcome in the subject.

Using the methods and compositions described herein it is possible to rapidly and quantitatively analyze the TME of a solid tumor to provide clinically relevant information based on phenotypic and functional analysis of tumor infiltrating lymphocytes (TILs) within the TME so as to determine the immune status within the TME. The characterization of TILs in the context of TME can be important for targeted immunotherapies. For example, a phenotypic subset analysis of TILs can be correlated with clinical outcomes and used as a treatment directing assay for immunotherapeutics. For example, increased CD8+ and CD56+ TIL density is predictive of positive response to immune-oncology treatment.

Similarly, the methods and compositions described herein facilitate the assessment of tumor-infiltrating NK, T cells (Tregs, CD8+ cytotoxic T cells, exhausted T cells), myeloid-derived suppressor cells (MDSC), and various subsets of dendritic cells (DCs) and macrophages (Mf) in the TME. These approaches further facilitate an evaluation of the proportion of immune modulatory receptor (IMR)/immune modulatory receptor ligand (IMR-L)-positive immune and tumor cells. The methods also allow for the analysis of activation/exhaustion markers, such as cytokines and granzyme expression.

Furthermore, the methods and compositions described herein can be used to determine the functional status of TILs, which can aid in predicting the clinical response of a given subject with a tumor. For example, using the approaches described herein it is possible to determine, for a given solid tumor sample, whether immune cells are activated or exhausted, and whether immune cells are being suppressed within the TME by Tregs, M2 macrophages, and MDSCs. Furthermore, the methods and compositions also facilitate the quantification of TIL and tumor cell (co-)expression profiles of targetable IMRs and their cognate ligands, which provides health care providers with information about whether a particular immunomodulator may positively impact the TME and facilitate a positive treatment outcome with the immune-modulator, either alone or in combination with another cancer drug. For example, the selection of a particular immunomodulator may reduce or eliminate the resistance a particular cancer cell may have to treatment with a particular cancer drug.

In general, the approaches described herein involve combining a single cell suspension of cells derived from a solid tumor with a plurality of labeling agents capable of binding a corresponding plurality of cell surface markers expressed on cancer cells and/or immune cells, and permitting the agents to simultaneously bind to cancer cells, immune cells or both cancer cells and immune cells present in the single cell suspension to produce labeled cells. The method includes determining the presence and/or amount of the labeled cells by cytometry thereby to determine the presence and/or amount of cancer cells, immune cells, or both cancer cells and immune cells present in the microenvironment of the solid tumor together as cell as phenotypic information about whether the cells are expressing certain phenotypic markers such as IMRs, IMR-Ls, and cell activation markers.

In certain embodiments, the method further includes the step of combining the cells with an immunomodulator, either alone or in combination with a cancer drug, and then determining whether immunomodulator impacts the expression of the cellular markers on the cancer and/or immune cells.

The methods and compositions described herein can be used to determine whether a subject with solid tumor is likely to respond to an immunomodulator. For example, a single cell suspension of cells derived from a solid tumor is combined with a plurality of labeling agents capable of binding a corresponding plurality of cell surface markers expressed on cancer cells and/or immune cells, and permitting the agents to simultaneously bind to cancer cells, immune cells, or both cancer cells an immune cells present in the single cell suspension to produce labeled cells. In addition, at least a portion of the single cell suspension of cells is contacted with an immunomodulator (either alone or in combination with a cancer drug), after which the labeled cells can be analyzed by cytometry to determine the presence and/or amount of cancer cells, immune cells or both cancer cells and immune cells present in the solid tumor. In addition, the effect of the immunomodulator on the cellular markers on or in the cancer cells, immune cells or both the cancer cells and immune cells, can be determined. This information can be used to determine whether a subject is likely to respond positively to the immunomodulator.

The methods and compositions described herein can be used in the treatment of a solid tumor in a subject in need thereof, whereby an effective amount of an immunomodulator selected by the approaches described herein is administered to the subject thereby to treat the solid tumor. The immunomodulator is selected by using a method comprising the steps of (a) combining a single cell suspension of cells derived from the solid tumor with a plurality of labeling agents capable of binding a corresponding plurality of cell surface markers expressed on cancer cells and/or immune cells, and permitting the agents to simultaneously bind to the cancer cells, immune cells or both cancer and immune cells present in the single cell suspension to produce labeled cells; (b) combining at least a portion of the single cell suspension of cells with an immunomodulator; and (c) determining (i) the presence and/or amount of the labeled cells by cytometry thereby to determine the presence and/or amount of cancer cells, immune cells, or both cancer cells and immune cells present in the solid tumor and (ii) the effect of the immunomodulator on the cellular markers on or in the cancer cells, immune cells or both the cancer cells and immune cells thereby to determine whether the subject is likely to respond to the immunomodulator.

The following sections describe in more detail how the methods and compositions described herein can be used to analyze the TME of a solid tumor and how it may be possible to determine whether a subject may respond favorably to a given immunomodulator.

### I. Cell Types

It is appreciated that the TME comprises both tumor cells and immune cells described herein.

(a) Tumor Cells

The term “tumor cell” is used herein interchangeably with “cancer cell.” Tumor cell types that can be interrogated using the methods and compositions described herein include epithelial-derived cells, endothelial-derived cells, and mesenchymal-derived cells.

Tumors that can be interrogated using the approaches described herein include, without limitation, anal cancer, bladder cancer, bowel cancer (large and small bowel), brain cancer, breast cancer, cancer of the oral cavity, cervical cancer, esophageal cancer, Fallopian tube cancer, head and neck cancer, colon cancer, colorectal cancer, lung cancer, ovarian cancer, pancreatic cancer, peritoneal cancer, prostatic cancer, rectal cancer, skin cancer, stomach cancer, testicular cancer, thymus cancer, thyroid cancer, urinary tract cancer, uterine cancer, and vulvar cancer.

Exemplary tumors include, for example, ovarian carcinoma (serous cystadenocarcinoma, mucinous cystadenocarcinoma, endometrioid carcinoma), ovarian granulosa cell tumor, Fallopian tube adenocarcinoma, peritoneal carcinoma, uterine (endometrial) adenocarcinoma, sarcomatoid carcinoma, cervical cell carcinoma, endocervical adenocarcinoma, vulvar carcinoma, breast carcinoma, primary and metastatic (ductal carcinoma, mucinous carcinoma, lobular carcinoma, malignant phyllodes tumor), head and neck carcinoma, oral cavity carcinoma including tongue, primary and metastatic, esophageal carcinoma, adenocarcinoma, gastric adenocarcinoma, primary small bowel carcinoma, colonic adenocarcinoma, primary and metastatic (adenocarcinoma, mucinous carcinoma, large cell neuroendocrine carcinoma, colloid carcinoma), appendiceal adenocarcinoma, colorectal carcinoma, rectal carcinoma, anal carcinoma (squamous, basaloid), carcinoid tumors, primary and metastatic (appendix, small bowel, colon), pancreatic carcinoma, liver carcinoma (hepatocellular carcinoma, cholangiocarcinoma), metastatic carcinoma to the liver, lung cancer, primary and metastatic (squamous cell, adenocarcinoma, adenosquamous carcinoma, giant cell carcinoma, nonsmall cell carcinoma, non small cell lung cancer (NSCLC), small cell carcinoma neuroendocrine carcinoma, large cell carcinoma, bronchoalveolar carcinoma), renal cell (kidney) carcinoma, primary and metastatic, urinary bladder carcinoma, primary and metastatic, prostatic adenocarcinoma, primary and metastatic, brain tumors, primary and metastatic (glioblastoma, multiforme, cerebral neuroectodermal malignant tumor, neuroectodermal tumor, oligodendroglioma, malignant astrocytoma), skin tumors (malignant melanoma, sebaceous cell carcinoma), thyroid carcinoma (papillary and follicular), thymic carcinoma, shenoidal carcinoma, carcinoma of unknown primary, neuroendocrine carcinoma, testicular malignancies (seminoma, embryonal carcinoma, malignant mixed tumors), and others.

(b) Immune Cells

Immune cells that can be detected and analyzed according to the present invention include lymphocytes (e.g., T cells (e.g., CD4+ T cells, CD8+ T cells, Tregs), B-cells, and natural killer cells), myeloid cells (e.g., dendritic cells, macrophages, and myeloid-derived suppressor cells (granulocyte and monocyte derived)).

### II. Sample Processing and Cell Counting

Solid tumor samples can be obtained from a subject and processed into a single cell suspension of cells by any means known in the art. As used herein, a “single cell suspension” is a suspension of one or more cells in a liquid sample, where the cells are predominantly in the form of single cells rather than in clusters or aggregates of cells. In certain embodiments, the single cells represent 60%, 70%, 80%, 90% or 95% of the cells in the cell suspension.

In certain embodiments, a solid tumor is manually and/or enzymatically digested. For example, a solid tumor sample may be cut into smaller pieces and subjected to enzymatic digestion using trypsin, collagenase, DNAse, dispase, and/or hyaluronidase. In certain embodiments, digested tissue is subjected to filtering to remove larger, undigested pieces.

In addition or alternatively to the above, tissue dissociation can be performed using an automated tissue homogenizer such as gentleMACS™ Octo Dissociator (Miltenyi Biotec GmbH, Bergish Gladbach, Germany). Cells may be filtered to remove undigested tissue, for example, using a 70 μM filter.

The resulting single cell suspension can contain all of the cell types present in the tumor microenvironment, including epithelial cells, endothelial cells, mesenchymal cells, stromal cells, tumor/cancer cells and immune cells.

Following dissociation, cells can be washed, pelleted, resuspended, and/or counted. Cells can be counted using any means known in the art, for example, using a manual cell counter or an automated cell counter. For example, for solid tumors, a nucleated cell count can be obtained using an automated cell counter that uses, e.g., bright field imaging and/or fluorescence imaging (e.g., dual-fluorescence imaging). Exemplary automated cell counters include the Nexcelom Cellometer 2000 (Nexcelom, Lawrence, Mass.), the Countess II FL Automated Cell Counter (ThermoFisher, Waltham, Mass.), and the TC20™ Automated Cell Counter (BioRad, Hercules, Calif.). For whole blood, bone marrow, PBMCs, or BMMCs, an automated cell counter such as a coulter counter can be used, e.g., a Beckman Coulter Act2 Diff (Beckman Coulter, Inc., Brea, Calif.).

After the approximate number of cells in the sample has been determined, cells can be pelleted and resuspended in a buffer at a desired concentration based upon the cell count. Buffers suitable for use include RPMI1640+10% FBS+1% Penicillin Streptomycin and or RPMI1640+10% FBS or 1×PBS+0.5% BSA. Cells can be resuspended at a concentration of from about 0.5 to about 5×10⁶ cells/mL, e.g., from about 0.5 to about 1×10⁶ cells/mL, from about 0.5 to about 2×10⁶ cells/mL, from about 0.5 to about 3×10⁶ cells/mL, from about 0.5 to about 4×10⁶ cells/mL, from about 1 to about 2×10⁶ cells/mL, from about 1 to about 3×10⁶ cells/mL, from about 1 to about 4×10⁶ cells/mL, from about 1 to about 5×10⁶ cells/mL, from about 2 to about 3×10⁶ cells/mL, from about 2 to about 4×10⁶ cells/mL, from about 2 to about 5×10⁶ cells/mL, from about 3 to about 4×10⁶ cells/mL, from about 3 to about 5×10⁶ cells/mL, from about 4 to about 5×10⁶ cells/mL. In certain embodiments, the cells are resuspended at a concentration of from about 1.2 to about 2.4×10⁶ cells/mL, from about 1.2 to about 2×10⁶ cells/mL, from about 1.2 to about 1.5×10⁶ cells/mL, from about 1.5 to about 2.4×10⁶ cells/mL, from about 1.5 to about 2×10⁶ cells/mL.

### III. Cellular Analysis

In order to analyze the cells initially disposed with the TME, the cells once converted into a single cell suspension are combined with a plurality (for example, 5, 6, 7, 8, 9, 10, 11, 20, 13, 14, 15, 16, 17, 18, 19, 20, 25, 30 or more) labeling agents that bind to select cell surface markers so as to determine whether cells are cancer cells or immune cells or sub-types, and/or whether such cells or sub-types express, for example, IMRs, IMR-Ls or cell activation markers.

Specifically, cells can be plated and stained using a vital dye (e.g., the amine reactive dye Alexa750) to distinguish live versus dead cells. Cells can be washed in stain buffer (e.g., 1×PBS+0.5% BSA). The washing step can be automated using, for example, a Biotek ELx450 deep well plate washer. Cells can be fixed and permeabilized using methods standard in the art, e.g., using the FOXP3/Transcription Staining Buffer Kit from eBioscience (ThermoFisher, Waltham, Mass.). Cells can then be washed in stain buffer (e.g., 1×PBS+0.5% BSA) and stained with one or more labeling agents to detect one or more cellular markers. Methods of staining cells will depend upon the labeling agents used.

(a) Cell Surface Markers

The methods disclosed herein can be used to detect any cell surface marker, such as a cancer cell marker, an activation marker or an IMR or IMR-L marker.

Cell type markers are proteins or other molecules that are present in or on specific cell types, e.g., cancer cells or immune cells. In certain embodiments, the presence of a specific cancer cell marker can indicate the type of cancer. In certain embodiments, the presence of a specific cancer cell marker provides a target for cancer treatment. Exemplary cancer cell markers include CD44, CD47, CD49f, CD271, CD326, cytokeratin (intracellular), E-cadherin, and/or vimentin. In certain embodiments, the presence of a specific immune cell marker identifies the immune cell as a certain immune cell type or subtype. Exemplary immune cell markers are provided in TABLE 1 below.

TABLE 1

| Populations Identified | Cell Markers |
|---|---|
| White blood cells | CD45(+) |
| T cells | CD3(+) |
| CD4+ T cells | CD3(+)/CD4(+) |
| CD8+ T cells | CD3(+)/CD8(+) |
| B cells | CD19(+) |
| NK cells | CD56(+) |
| Monocytes | CD14(+) |
| Non-classical | CD16(+) |
| monocytes | |
| Treg | CD3(+)/CD4(+)/CD25(+)/FoxP3(+) |
| M1 macrophage | CD14(+)/HLA-DR(+)/CD80(+)/CD163(−) |
| M2 macrophage | CD14(+)/HLA-DR(+)/CD80(−)/CD163(+) |
| Myeloid dendritic | CD3(−)/CD19(−)/CD56(−)/CD14(−)/HLA- |
| cell | DR(+)/CD11c(+)/CD123(−) |
| Plasmacytoid | CD3(−)/CD19(−)/CD56(−)/CD14(−)/HLA- |
| dendritic cell | DR(+)/CD11c(−)/CD123(+) |
| Granulocytic-Myeloid | CD33(+)/CD11b(+)/CD14(−)/CD15(+)/HLA- |
| derived supressor cell | DR(−) |
| Monocytic-Myeloid | CD33(+)/CD11b(+)/CD14(+)/CD15(−)/HLA- |
| derived supressor cell | DR(−) |
| Immature-Myeloid | CD33(+)/CD11b(+)/CD15(−)/CD14(−)/HLA- |
| derived | DR(−) |
| supressor cell | |

Activation markers can be proteins or other molecules. The presence of an activation marker in or on a cancer cell or immune cell is indicative that a certain pathway (e.g., an immune pathway) is active. Exemplary activation markers include CD25, CD26, CD27, CD28, CD38, CD40, CD44, CD62L, CD69, CD80, CD86, CD95, CD95L, CD127, CCR7 (CD197), and/or functional markers (e.g., IFNγ, TNFα, and/or other cytokines and/or Granzyme B.

Exemplary IMR or IMR-L markers include PD-1 (CD279), PD-L1 (CD274), CTLA-4 (CD152), LAGS (CD223), OX40 (CD134), TIM3 (CD366), GITR (CD357), 4-1BB (CD137), KIR (CD158B), 2B4 (CD244), ICOS (CD278), IDO, TIGIT, CD73, CD39, CD172a (SIRPa), B7H4 (B7S1), VISTA (B7-H5), CD355 (CRTAM), KLRG1, CD160 (BY55, NK1, NK28), CD30 (TNFRSF8), CD224 (GGT1), CD226, CD272 (BTLA), and/or CD115 (CSF-1R).

In certain embodiments, other markers of signaling can be detected according to the methods herein. In certain embodiments, the method includes detecting a receptor or transporter (e.g., CD3, CD4, CD5, CD8, CD11b, CD11c, CD14, CD15, CD16, CD19, CD20, CD25, CD27, CD28, CD31, CD34, CD38, CD45, CD45RA, CD45RO, CD56, CD69, CD71, CD80, CD86, CD90, CD117, CD123, CD133, CD135, CD235, Cytokaritin, EPCAM, FOXP3, HLA-DR, IgD, IgG, IgM, MDR1, ABCG2), a DNA damage or apoptosis signaling molecule (e.g., Bcl-2, Bcl-xL, Cytochrome C, Caspase 3 or 8, cPARP, annexinV, DNMT1, 3a, 3b, p-H2AX, p-53BP1, p-ATM, p-DNA-PKcs, p-p53, P53, p21, p-Chk2, p-RPA2, p-BRCA1), an immune signaling molecule (e.g., p-Akt, p-Blnk, p-Erk, p-Gsk3b, p-Lyn, p-NFkB, p-Plcg2, p-S6, p-Stat5, p-Syk, p-SLP-76, p-ZAP-70, p-Lck, p-CD3z, p-Vav, p-Lat, p-Pyk2), a differentiation, maturation and/or cytokine/chemokine response signaling molecule (e.g., p-Stat1, p-Stat3, p-Stat4, p-Stat5, p-Stat6, p-Erk, p-p38, p-NFkB, pRelB), an intracellular cytokine (e.g., IL-2, IL-4, IL-6, IL-8, IL-10, IL17 A, IFNa, IFNg, TNFa), a measure of cytotoxic effector function (e.g., CD107a, Granzyme, Perforin, Annexin V), a PKC, CA⁺⁺ signaling molecule (e.g., p-Akt p-Erk, p-PLCg2, p-PKCa p-S6, p-p38), a survival, proliferation, cell cycle and pattern recognition receptor signaling molecule (e.g., p-Akt p-NFkB p-S6, IkB, p-Erk p-p38, Cyclin A2, Cyclin B1, p-CDK1, p-HH3, p-MK2, p21, p-CREB, p-c-JUN).

In certain embodiments, a panel of cellular markers is measured to identify cell type, IMR and/or IMR-L, and or/an exhaustion marker. An exemplary panel of cellular markers is shown in TABLE 2.

TABLE 2

| Panel | Main population | Subsets | CD and other markers |
|---|---|---|---|
| T cell | CD4+ T cells | Naïve cells | CD127hi CR25lo |
| (functional) | | Memory cells | CD45RA− |
| panels | | Treg cells | CD25hi CD127lo or CD25hi |
| | | | FoxP3+ |
| | | Cytokine (+) | IFN-g+, TNF+, etc. |
| | CD8+ T cells | Naïve cells | CD127hi CR25lo |
| | | Cytokine (+) | IFN-g+, TNF+, etc. |
| | | Granzyme B+ | Granzyme B+ |
| | NK cells | CD56var | Live CD56var |
| | | CD3− NK | CD3− |
| | | CD3+ NKT | CD3+ |
| | | Cytokine (+) | IFN-g+, TNF+, etc. |
| | | Granzyme B+ | Granzyme B+ |
| | Gamma delta (gd) T | | Live CD3+ gdTCR+ |
| | cells | Cytokine (+) | IFN-g+, TNF+, etc. |
| | | Granzyme B+ | Granzyme B+ |
| Checkpoint | | | TIGIT, CD137, CD223, |
| Receptors, | | | CD274, CD279, CD357, |
| Exhaustion and | | | CD366; HLADR, CD25, |
| Activation | | | CD45RA, CD197 |
| Markers | MDSC | G-derived | CD11b+ CD14− CD15+ (or |
| Myeloid-derived | | | CD66b+) |
| suppressor cells | | M-derived | CD11b+/CD14hi/HLADR1o/ |
| (MDSC), | | | CD15− |
| Monocyte and | | MDSC immature | Lin−/CD33+/HLADRlo |
| Dendritic cell | | progenitors | |
| (DC) panels | Monocytes/ | | Live CD33+/CD14+/CD11c+/ |
| | macrophages | | CD11b+/CD16+−/CD15−/ |
| | | | CD123− |
| | | M1: | CD68+/CD80+/CCR7(CD197)+ |
| | | M2: | CD68+/CD163+/CD206+ |
| | | Nonclassical | CD14hi-lo/CD16+− |
| | | monocytes | |
| | | TAM and M2 | HLADR-lo, PD-L1+, CD33+, |
| | | markers | IDO+, CD163+, CD206+ |
| | Myeloid DC | | LIN2−*/HLADR+/CD11chi/ |
| | | | CD123−*/lo |
| | | | *CD3− CD19− CD56− CD14− |
| | | type 1 & type 2 | CD16−/CD1c+ or CD16−/ |
| | | | CD1c-lo |
| | | CD16+ DC & | CD16+ or CD141+ |
| | | CD141+ DCs | |
| | Plasmacytoid DCs | | LIN−/HLADR−/CD11c−/ |
| | | | CD16−/CD123+ |

For example, in certain embodiments, at least 5, at least 6, at least 7, at least 8, at least 9, at least 10, at least 11, at least 12, at least 13, at least 14, at least 15, at least 16, at least 17, at least 18, at least 19, at least 20 or more different cell surface markers are detected at the same time. In certain embodiments, between 5 and 19, between 6 and 18, between 7 and 17, between 8 and 16, between 9 and 15, between 10 and 14, between 10 and 13, between 11 and 12, between 5 and 14, between 6 and 14, between 7 and 14, between 8 and 14, between 9 and 14, between 10 and 14, between 11 and 14, between 12 and 14, between 13 and 14, between 5 and 16, between 6 and 16, between 7 and 16, between 8 and 16, between 9 and 16, between 10 and 16, between 11 and 16, between 12 and 16, between 13 and 16, between 14 and 16, or between 15 and 16 different cell surface markers are detected at the same time. In certain embodiments of the methods described herein, at least 14 different cell surface markers are detected at the same time.

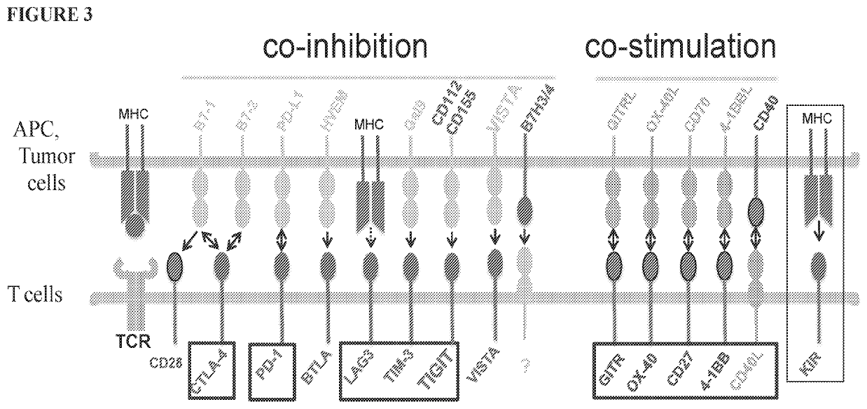

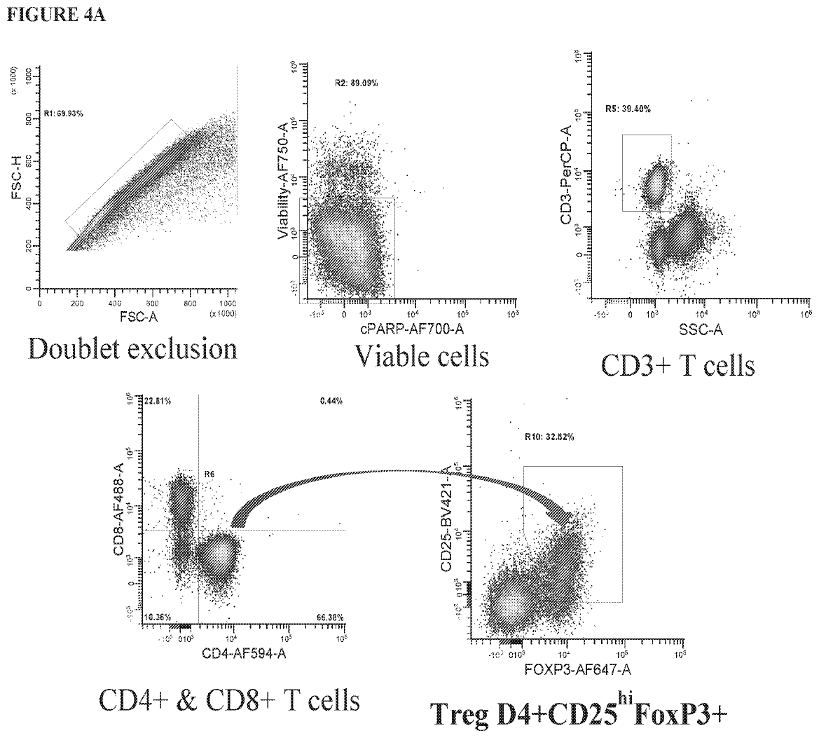

In certain embodiments, receptor-ligand interactions between the labeled cells can be detected, for example, interactions between a checkpoint inhibitor and its cognate ligand. Exemplary receptor-ligand pairs are depicted in FIG. 3. For example, the receptor-ligand interactions can be selected from the interactions between PD-1 and PD-L1, CTLA-4 and B7-1 and/or B7-2, TIM-3 and Gal9, TIGIT/CD112-CD155, GITR and GITRL, OX-40 and OX40L, CD-27 and CD70, 4-1BB and 4-1BBL, LAGS/MHC, KIR?MHC, and/or CD-40L and CD40.

(b) Labeling Agents For Combination with Single Cell Suspensions

It is contemplated that the single cell suspensions, once prepared, can be distributed between a plurality of receiving vessels, for example, wells in a multi-well plate, e.g., a 96 or 364 well plate, and prepared for cytometric analysis.

Cell plating and subsequent handling steps can be performed automatically, e.g., with a Hamilton robotics liquid handlers (Hamilton Company, Reno, Nev.). Cells can be fixed and permeabilized prior to being exposed to a labeling agent using any method known in the art. In certain embodiments, cells are exposed to a labeling agent without fixation and permeabilization, or prior to fixation and permeabilization, if the labeling agent is sensitive to fixation. In certain embodiments, the labeling agent comprises a binding agent (e.g., a member of a binding complex, for example, an antibody, protein, aptamer, avimer, Adnectin and Affibody® ligand, a member of a ligand receptor pair, small molecule inhibitor) coupled, for example, covalently coupled, to a label, wherein the binding agent binds to a cell surface marker (e.g., cancer cell marker, an activation marker or an immune modulatory receptor (IMR) or IMR-ligand (IMR-L) marker). In certain embodiments, the cells are exposed to a binding agent (e.g., an antibody) that binds to a cell surface marker to form a cell surface marker/binding agent complex, and the cell surface marker/binding agent complex is exposed to a labeling agent that binds to the cell surface marker/binding agent complex. In certain embodiments, oligonucleotide conjugates (i.e., oligonucleotide labels) are used, in which a binding agent (e.g., an antibody) is conjugated to a first oligonucleotide, and a second oligonucleotide complementary (i.e., capable of binding (hybridizing)) to the first oligonucleotide is conjugated directly or indirectly to one or more labels (e.g., one or more fluorophores). Annealing of the first and second oligonucleotides connects the binding agent to the one or more labels. The annealed oligonucleotide conjugate, which comprises the binding agent-label conjugate can then be used in cytometry applications (e.g., flow cytometry).

(c) Binding Agents

Binding agents suitable for use according to the methods herein include any substance that can bind preferentially to the cell surface markers (e.g., cancer cell marker, an activation marker or an immune modulatory receptor (IMR) or IMR-ligand (IMR-L) marker) described herein. For example, binding agents can include antibodies (e.g., monoclonal antibodies) proteins, peptide aptamers, avimers, Adnectins and Affibody® ligands, a member of a ligand receptor pair, and a small molecule inhibitor.

Exemplary, binding agents can include a CD44 binding agent, a CD47 binding agent, a CD49f binding agent, a CD271 binding agent, a CD326 binding agent, a cytokeratin binding agent, an E-cadherin binding agent, a vimentin binding agent, a CD25 binding agent, a CD26 binding agent, a CD27 binding agent, a CD28 binding agent, a CD38 binding agent, a CD40 binding agent, a CD44 binding agent, a CD62L binding agent, a CD69 binding agent, a CD80 binding agent, a CD86 binding agent, a CD95 binding agent, a CD95L binding agent, a CD127 binding agent, a CCR7 (CD197) binding agent, a IFNγ binding agent, a TNFα binding agent, a Granzyme B binding agent, a PD-1 (CD279) binding agent, a PD-L1 (CD274) binding agent, a CTLA-4 (CD152) binding agent, a LAG3 (CD223) binding agent, an OX40 (CD134) binding agent, a TIM3 (CD366) binding agent, a GITR (CD357) binding agent, a 4-1BB (CD137) binding agent, a KIR (CD158B) binding agent, a 2B4 (CD244) binding agent, a ICOS (CD278) binding agent, an IDO binding agent, a TIGIT binding agent, a CD73 binding agent, a CD39 binding agent, a CD172a (SIRPa) binding agent, a B7H4 (B7S1) binding agent, a VISTA (B7-H5) binding agent, a CD355 (CRTAM) binding agent, a KLRG1 binding agent, a CD160 (BY55, NK1, NK28) binding agent, a CD30 (TNFRSF8) binding agent, a CD224 (GGT1) binding agent, a CD226 binding agent, a CD272 (BTLA) binding agent, and/or a CD115 (CSF-1R) binding agent.

Antibodies suitable for use in accordance with the methods herein include an anti-CD44 antibody, an anti-CD47 antibody, an anti-CD49f antibody, an anti-CD271 antibody, an anti-CD326 antibody, an anti-cytokeratin antibody, an anti-E-cadherin antibody, an anti-vimentin antibody, an anti-CD25 antibody, an anti-CD26 antibody, an anti-CD27 antibody, an anti-CD28 antibody, an anti-CD38 antibody, an anti-CD40 antibody, an anti-CD44 antibody, an anti-CD62L antibody, an anti-CD69 antibody, an anti-CD80 antibody, an anti-CD86 antibody, an anti-CD95 antibody, an anti-CD95L antibody, an anti-CD127 antibody, an anti-CCR7 (CD197) antibody, an anti-IFNγ antibody, an anti-TNFα antibody, an anti-Granzyme B antibody, an anti-PD-1 (CD279) antibody, an anti-PD-L1 (CD274) antibody, an anti-CTLA-4 (CD152) antibody, an anti-LAG3 (CD223) antibody, an anti-OX40 (CD134) antibody, an anti-TIM3 (CD366) antibody, an anti-GITR (CD357) antibody, an anti-4-1BB (CD137) antibody, an anti-KIR (CD158B) antibody, an anti-2B4 (CD244) antibody, an anti-ICOS (CD278) antibody, an anti-IDO antibody, an anti-TIGIT antibody, an anti-CD73 antibody, an anti-CD39 antibody, an anti-CD172a (SIRPa) antibody, an anti-B7H4 (B7S1) antibody, an anti-VISTA (B7-H5) antibody, an anti-CD355 (CRTAM) antibody, an anti-KLRG1 antibody, an anti-CD160 (BY55, NK1, NK28) antibody, an anti-CD30 (TNFRSF8) antibody, an anti-CD224 (GGT1) antibody, an anti-CD226 antibody, an anti-CD272 (BTLA) antibody, and/or an anti-CD115 (CSF-1R) antibody.

(d) Labeling Agents

(i) Fluorophores (Including Visible Fluorophore Labels and Infrared Fluorophore Labels)

Fluorophores suitable for use according to the methods herein include, but are not limited to, Cy5.5, Cy5 and Cy7 (GE Healthcare); AlexaFluor 488, AlexaFluor 594, AlexaFluor 647, AlexaFluor660, AlexaFluor680, AlexaFluor700, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); and ADS780WS, ADS830WS, and ADS832WS (American Dye Source) and Kodak X-SIGHT 650, Kodak X-SIGHT 691, Kodak X-SIGHT 751 (Carestream Health), PE, PE-Cy7, PerCP, PerCP-Cy5.5, FITC, BV421, BV510, BV605.

(ii) Heavy Metal Labels

Heavy metal labels, such as lanthanides, are used in certain embodiments of the methods described herein, e.g., mass cytometry. Lanthanides include, but are not limited to, lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu). In certain embodiments, Lanthanide isotopes are used, including, for example, ¹³⁹La, ¹⁴¹Pr, ¹⁴²Nd, ¹⁴⁴Nd, ¹⁴⁵Nd, ¹⁴⁶Nd, ¹⁴⁷Nd, ¹⁴⁷Sm, ¹⁵²Sm, ¹⁵¹Eu, ¹⁵³Eu, ¹⁵⁶Gd, ¹⁵⁹Tb, ¹⁶⁴Dy, ¹⁶⁵Ho, ¹⁶⁶Er, ¹⁶⁹Tm, ¹⁷¹Yb, ¹⁷⁴Yb, and ¹⁷⁶Yb.

(e) Cytometry

(i) Flow Cytometry

In certain embodiments, labeled cells are analyzed using flow cytometry. Once labeled cells are obtained using, e.g., a staining procedure described herein, the labeled cells are analyzed by flow cytometry. Exemplary flow cytometers useful for the methods herein include, for example, the Attune NxT flow cytometer (ThermoFisher, Waltham, Mass.), the CytoFLEX flow cytometer (Beckman Coulter, Indianapolis, Ind.), the FACSVerse or FACSCanto II flow cytometers (Becton Dickinson, San Jose, Calif.), or the Aurora flow cytometer (Cytek, Oakland, Calif.) that are capable of measuring multiple, e.g., 16, fluorochromes at the same time. Data capture from the flow cytometer can be analyzed using, for example, the standard settings of the flow cytometer.

In certain embodiments, prior to acquisition of the flow cytometry data, information such as sample ID, labeling agent, and flow cytometer instrument settings and acquisition parameters are incorporated into a plate layout file using Ryvett software from Qognit, Inc. (Qognit, San Carlos, Calif.). This plate layout file can be imported into the flow cytometry software prior to acquisition. After the flow cytometry data is generated, flow cytometry standard (FCS) files can be analyzed and gated in Ryvett software utilizing a desired gating routine. Following manual review of auto-gating, raw data and calculated metrics can be exported to a CSV file utilizing pre-defined criteria and data extraction routines.

In certain embodiments, flow cytometry data can be analyzed using gating in WinList (Software House, Topsham, Me.), Ryvett (Qognit, Redwood City, Calif.), FlowJo (FloJo LLC, Ashland, Oreg.) or Kaluza (Beckman Coulter, Indianapolis, Ind.).

Using this software, the cell population in the TME can be interrogated to determine what cancer and immune cells are present in the TME, their status (e.g., inactive, active or exhausted), and whether the cells are expressing one or more IMRs or IMR-Ls.

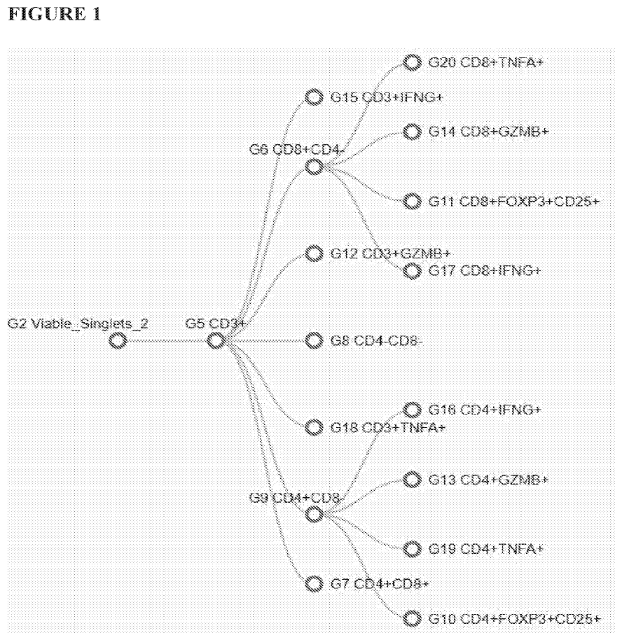

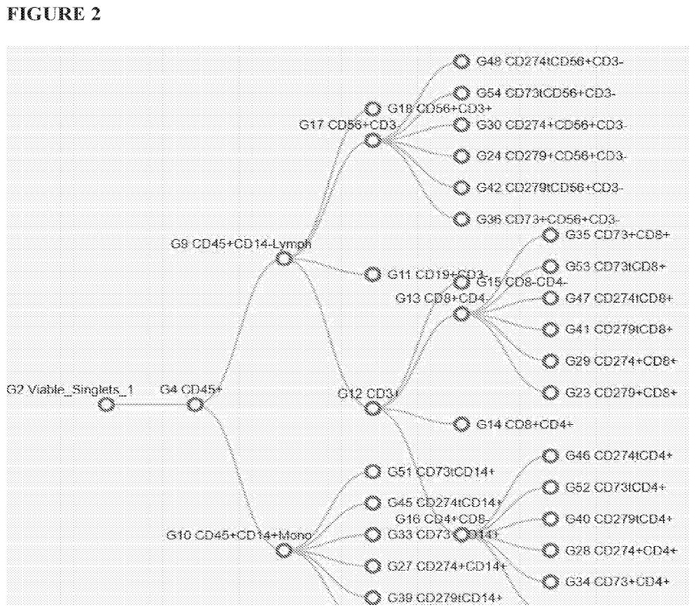

As is understood in the art, a gating tree can be used to analyze cell populations from a flow cytometric experiment and segregate them into more discrete populations as the gating tree branches. FIGS. 1 and 2 depicts a gating tree for automated metrics extraction from pre-determined gating routines for the identification of Tregs and expression of Granzyme B or cytokines in a Treg staining panel. Each of the gates (defined as G4, G9, G10, G17, etc. on FIGS. 1 and 2) are composed of multiple regions which are combined to make a gating scheme utilizing Boolean logic (e.g., “and”, “or”, and “not” statements) to include, exclude, or combine cell populations displayed in a gate.

In addition to determining the presence of a given cell surface markers (e.g., cell-type markers, immune modulatory receptors (IMRs) or IMR-ligands (IMR-L) on a cell, the methods described herein can include quantitatively measuring the amount of such a marker. For example, the amount of a marker can be directly measured as an equivalent number of reference fluorophores (ERFs). See, e.g., Gaigalas et al. (2016) JOURNAL OF RESEARCH OF THE NATIONAL INSTITUTE OF STANDARDS AND TECHNOLOGY 121:264-281. In certain embodiments, a subject will be determined to be likely to respond to an immunomodulator if the amount of marker (e.g., IMR or IMR-L, activation, and/or exhaustion marker measured exceeds a certain threshold.

(ii) Mass Cytometry

In certain embodiments, labeled cells are analyzed using mass cytometry. Mass cytometry combines flow cytometry and mass spectrometry. In contrast to flow cytometry, which differentiates signals by measuring the fluorescent spectra of different reporters, mass cytometry uses probes (e.g., antibodies) coupled to stable heavy metal isotopes, using a mass cytometer such as a Cytometry by Time-Of-Flight (CyTOF®) system (Fluidigm, San Francisco, Calif.). (Bandura et al. (2009) ANAL CHEM. 81:6813-6822; Bjornson et al. (2013) CURRENT OPINION IN IMMUNOLOGY 25:484-494.) Once labeled cells are obtained using, e.g., using methods known in the art, the labeled cells are analyzed by mass cytometry. Exemplary mass cytometers useful for the methods herein include, for example, Helios® (Fluidigm, San Francisco, Calif.). Data capture from the mass cytometer can be analyzed using, for example, the mass cytometer software, e.g., CyTOF® software v. 7.0 (Fluidigm, San Francisco, Calif.).

In certain embodiments, mass cytometry data can be analyzed using conditional-Density Resampled Estimate of Mutual Information (DREMI), which optionally can be coupled with conditional-Density Rescaled Visualization (DREVI) (Krishnaswamy et al. (2014) SCIENCE 346(6213): 1250689).

Accordingly, like flow cytometry, mass cytometry can be used to interrogate the cell population in the TME to determine what cancer and immune cells are present in the TME, their status (e.g., inactive, active or exhausted), and whether the cells are expressing one or more IMRs or IMR-Ls. In addition, mass cytometry can be used to perform single cell genomic sequencing to reveal, for example, mutations (e.g., somatic mutations) in single cells (e.g., an immune cell or a cancer cell).

(iii) Image Cytometry

In certain embodiments, labeled cells are analyzed using image cytometry. Image cytometry can be used to measure many of the same parameters as flow cytometry, but in addition, includes three-dimensional imaging using automated microscopy and computational image processing and analysis, which allows for the acquisition and identification of tens of thousands of cellular events based on fluorescent and/or morphological parameters. Accordingly, in contrast to flow cytometry, image cytometry can also evaluate cellular events by their real images. A summary of imaging cytometry is provided by Barteneva et al. (2012) J HISTOCHEM CYTOCHEM 60(10): 723-733.

Exemplary imaging cytometers include the FlowSight® Imaging Flow Cytometer and the ImageStream®^{X} Mk II Imaging Flow Cytometer (Luminex Corp., Austin, Tex.).

In certain embodiments, image cytometry is used to interrogate the cell population in the TME to determine what cancer and immune cells are present in the TME, their status (e.g., inactive, active or exhausted), and whether the cells are expressing one or more IMRs or IMR-Ls. In addition, in certain embodiments, image cytometry is used to assess morphological characteristics of cells (e.g., cancer and/or immune cells), co-localization of two proteins, binding of two cells (e.g., cancer and/or immune cells), visualization of immune synapse formation, and nuclear translocation. In addition, image cytometry can be used to perform single cell genomic sequencing to reveal, for example, mutations (e.g., somatic mutations) in single cells (e.g., an immune cell or a cancer cell). Image cytometry can also be used in conjunction with laser capture microdissection (LCM) for laser ablation mass spectrometry and for proteomic and genomic or mRNA analysis.

(iv) Single Cell Technologies (SCTs)

Single cell technologies (SCTs) can also be used in accordance with the methods disclosed herein to assess individual cells in the TME and their interactions with other cells under different conditions (e.g., in the presence of an immunomodulatory and/or a cancer drug). Single cells from a TME can be isolated an manipulated using a number of different methods, including fluidic-based, physical based, electric filed-driven (e.g., dielectrophoreses (DEP), optoelectronic tweezers (OET) and optical techniques such as optical tweezers. (See, e.g., Skelley et al. (2009) NAT. METHODS 6:147-152; Thieleche et al. (1999) IEEE ENG. MED. BIOL. MAG. 18:48-52; Taff et al. (2005) ANAL CHEM. 77:7976-7983; Juan et al. (2011) NAT. PHOTONICS 5:349-356; and Mirsaidov et al. (2008) LAB CHIP 8:2174-2181.

In certain embodiments, SCT is performed using a microfluidic chip. For example, SCT can be performed using a hydrodynamic-based microfluidic chip. In other embodiments, the dielectrophoretic digital sorting method uses a semiconductor controlled array of electrodes in a microfluidic chip to trap single cells in dielectrophoretic (DEP) cages.

In certain embodiments, SCT is performed using a microfluidic slide (e.g., nCounter® by NanoString Technologies, Inc., Seattle, Wash.; Genesis System by Celsee®, Ann Arbor, Mich.).

Single cells can be analyzed using a number of techniques, including assessment of growth rate (Cermak et al. (2016) NAT. BIOTECH 34:1052-1059), measurement of cell membrane potential (Liu et al. (2017) NANO LETT. 17:2757-2764), assessment of the cell's genome and/or transcriptome (Horgan (2011) OBSTET. GYNAECOL. 13:189-195), proteomics (Horgan, (2011) supra), and mass spectrometry (Li et al. (2000) TRENDS BIOTECHNOL. 18:151-160). In certain embodiments, cells are assessed using one or more of the preceding SCT techniques to determine their cell type, their status (e.g., inactive, active or exhausted), and whether the cells are expressing one or more IMRs or IMR-Ls.

### V. Immunomodulators

The systems described above can also be used to determine whether the cells isolated from the TME respond to the addition an immunomodulator, either alone or in combination with a cancer drug.

Immunomodulators suitable for use herein include any substance capable of modulating an immune cell, for example, by activating the immune system to kill tumor cells or by removing an immune cell inhibitory signal from a tumor cell. In certain embodiments, cells are exposed to an immunomodulator prior to being combined with a labeling agent.

In certain embodiments, the immunomodulator is a checkpoint inhibitor. The checkpoint inhibitor may, for example, be selected from a PD-1 antagonist, PD-L1 antagonist, CTLA-4 antagonist, adenosine A2 A receptor antagonist, B7-H3 antagonist, B7-H4 antagonist, BTLA antagonist, KIR antagonist, LAGS antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MED14736, avelumab, and BMS 936559 (Bristol Myers Squibb Co.).

In certain embodiments, the immunomodulator is a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, the immunomodulator is administered in combination with an IDO inhibitor. Exemplary IDO inhibitors include 1-methyl-D-tryptophan (known as indoximod), epacadostat (INCB24360), navoximod (GDC-0919), and BMS-986205.

Other immunomodulators include, for example, anti-CD20 antibodies such as Arzerra® (ofatumumab, GlaxoSmithKine), Rituxan® (rituximab, Genentech, Biogen), and Mabthera® (rituximab, Roche); and anti-CD52 antibodies such as Campath® (alemtuzumab, Genzyme).

Additional antibody-based immunomodulators include those set forth in TABLE 3. For certain antibodies in TABLE 3, a type of cancer targeted by the antibody or antibody-drug conjugate is also indicated.

TABLE 3

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| pertuzumab | human epidermal growth factor | |
| | receptor 2 (Her2) | |
| trastuzumab | Her2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab; | | (NHL), chronic lymphocytic |
| ibritumomab (e.g., ⁹⁰Y- | | leukemia (CLL) |
| ibritumomab; | | B-cell NHL |
| tositumomab (e.g., ¹³¹T- | | pre-B ALL |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab; | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | (NSCLC) |
| dinutuximab; hu3F8; | disialoganglioside GD2 (GD2) | Neuroblastoma |
| hu14.18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 (LAG-3) | Breast, Hematological, |
| | | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox) | | Prostate |

Once one or more immunomodulators have been identified, that, either alone or in combination with a cancer drug (such as a compound discussed below) provide a positive outcome on the cancer and/or immune cells in a TME, the immunomodulator, either alone or in combination with a cancer drug, can be administered to the subject.

### IX. Pharmaceutical Compositions and Administration of Immunomodulators

For therapeutic use, the immunomodulator preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing an immunomodulator disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. A preferred route of administration is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, an immunomodulator, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the immunomodulator, and the disease being treated. Exemplary dosing frequencies are once per day, once per week and once every two weeks. A preferred route of administration is parenteral, e.g., intravenous infusion. In certain embodiments, an immunomodulator is lyophilized, and then reconstituted in buffered saline, at the time of administration.

### VI. Therapies

Once a suitable immunomodulator, either alone or in combination with a cancer drug, has been selected for a given subject, the subject can be treated in accordance with conventional health care practices. In particular, an effective amount of the immunomodulator, either alone or in a combination with an effective amount of another cancer drug, can be administered to the subject. The term “effective amount” as used herein refers to the amount of an active agent (e.g., an immunomodulator) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers that can be treated by the approaches described herein are described in Section I. In certain embodiments, the cancer is a metastatic cancer. In certain embodiments, the cancer is a refractory cancer.

As noted above, it is contemplated that the immunomodulator can be administered, either alone or on combination, with another cancer drug or therapeutic agent. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the immunomodulator is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

Exemplary cancer drugs that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

As used herein, the terms “subject” and “patient” are used interchangeably and refer to an organism to be treated by the methods and compositions of the present invention. Such organisms are preferably mammals (e.g., human, mouse, rat, guinea pig, dog, cat, horse, cow, pig, or non-human primate, such as a monkey, chimpanzee, baboon, and rhesus), and more preferably humans.

As used herein, the term “treating” includes any effect, for example, lessening, reducing, modulating, arresting, slowing the progression of, ameliorating or eliminating, that results in the improvement of the condition, disease, disorder, and the like, or ameliorating a symptom thereof. For example, treating a cancer or a tumor can mean reducing the growth of the cancer or tumor, modulating the cancer or tumor arresting the growth of the cancer or tumor, slowing the progression of the cancer or the growth of the tumor, ameliorating or eliminating the cancer or the growth of the tumor. Treating can be curing, improving, or at least partially ameliorating the disorder, e.g., cancer. In certain embodiments, treating is curing the disease, e.g., cancer. The term “disorder” refers to and is used interchangeably with, the terms disease, condition, or illness, unless otherwise indicated.

In the application, where an element or component is said to be included in and/or selected from a list of recited elements or components, it should be understood that the element or component can be any one of the recited elements or components, or the element or component can be selected from a group consisting of two or more of the recited elements or components.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

Unless defined otherwise, all technical and scientific terms used herein have the same meaning as commonly understood by one of ordinary skill in the art to which this invention belongs.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also include the specific quantitative value itself, unless specifically stated otherwise. As used herein, the term “about” refers to a ±10% variation from the nominal value unless otherwise indicated or inferred.

Where a molecular weight is provided and not an absolute value, for example, of a polymer, then the molecular weight should be understood to be an average molecule weight, unless otherwise stated or understood from the context.

As a general matter, compositions specifying a percentage are by weight unless otherwise specified. Further, if a variable is not accompanied by a definition, then the previous definition of the variable controls.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby.

### Example 1—Determining the Composition of a Tumor Microenvironment

This Example shows the ability of the claimed methods to determine the composition of a tumor microenvironment from lung, breast, and renal solid tumors utilizing both surface and intracellular markers. Tumors showing elevated levels of CD4+CD25^{hi}FoxP3+ Tregs suggest an immunosuppressive signature with tumor spreading, and thereby a poor prognosis and objective response, which can be useful in developing a treatment strategy for the patient.

In this example, lung, breast, and renal solid tumor samples were obtained and shipped to Pierian Biosciences in Miltenyi Tissue Transport Buffer in Therapak controlled rate shippers maintained at 2-8° C. Upon receipt, tumors were cut into 2-3 mm pieces using sterile disposable scalpels and placed into tumor dissociation buffer (RPMI1640 plus pen/strep). The tumor pieces were then mechanically dissociated into a single cell suspension using enzymes from Miltenyi's human tumor dissociation kit and Miltenyi's gentleMACS™ Octo Dissociator with heaters. The single cell suspension was subsequently filtered through a 70 μM filter and counted. The cell suspension was then centrifuged and resuspended at the desired cell concentration in RPMI1640+10% FBS or 1×PBS+0.5% BSA.

After resuspending cells in the appropriate buffer, 80 μL were plated into designated wells of a deep well 96-well plate (2 mL volume). Subsequently, cells were initially stained using the amine reactive dye Alexa750 for 15 min to distinguish live versus dead cells. Cells were then washed 2× in stain buffer (1×PBS+0.5% BSA) using a Biotek ELx450 deep well plate washer. Cells were subsequently fixed and permeabilized utilizing the FOXP3/Transcription Staining Buffer Kit from eBioscience according to the manufacturers recommendations for processing and staining cells in a deep-well plate. Cells were then washed 2× in stain buffer (1×PBS+0.5% BSA) using a Biotek ELx450 deep well plate washer. Cells were then stained with fluorescently labeled antibody cocktails to detect CD3+, CD4+, CD8+ and Tregs (CD4+CD25^{hi}FoxP3+). Stained cells were incubated for 1 h at ambient temperature in the dark. Cells were then washed 2× in stain buffer (1×PBS+0.5% BSA) using a Biotek ELx450 deep well plate washer prior to fixation in PFA at a final concentration of 1%.

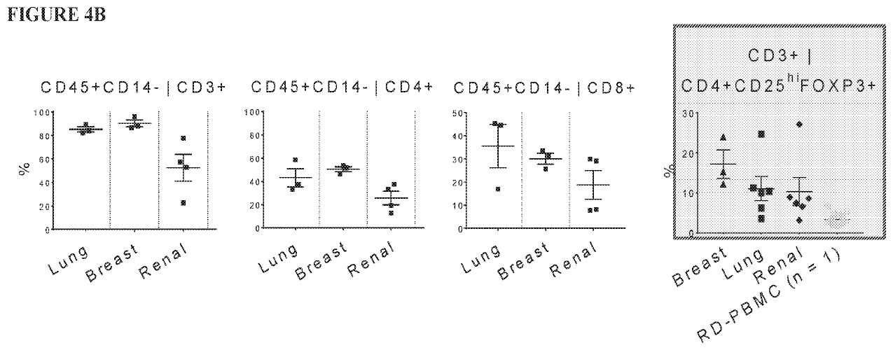

Stained cells were acquired directly from the 96-well deep well plate using a Thermo-Fisher Attune NxT 16-color flow cytometer. Prior to acquisition, all required information such as sample ID, antibody staining panels, and pre-determined and fixed flow cytometer instrument settings and acquisition parameters were incorporated into a 96 well plate layout using Ryvett software from Qognit, Inc. This plate layout file was imported into the Attune NxT software prior to acquisition. Post acquisition, FCS files were automatically analyzed and gated in Ryvett software utilizing pre-determined gating routines. Following manual review of auto-gating, raw data and calculated metrics were exported to a CSV file utilizing pre-defined criteria and data extraction routines. The results are shown in FIGS. 4A-B. FIG. 4A shows exemplary flow cytometry plots showing the detection of CD3+, CD4+, CD8+ and Tregs (CD4+CD25^{hi}FoxP3+). FIG. 4B provides a dot plot showing expression of CD3+ T cells (expressed as a percentage of CD45+ leukocytes), CD4+ and CD8+ T cells (expressed as a percentage of CD3+ T cells) and Tregs (expressed as a percentage of CD4+ T cells).

The results above show that the presence and amount of specific types of immune cells can be detected in the tumor microenvironment using the methods described herein. Understanding the composition of immune cells in the tumor microenvironment, e.g., ratios of CD4+/CD8+ cells and CD8+/Tregs, is important for determining the potential for a patient to respond to the prescribed immunotherapy.

### Example 2—Assessing Functional Capacity of Cells from Dissociated Tumors Through Surface and Intracellular Staining of Targets in Basal and Induced States

This Example shows the ability of the claimed methods to determine the functional capacity by measuring both basal and induced levels of functional readouts in identified immune cells from breast, lung, and renal solid tumors utilizing both surface and intracellular markers.

In this example, lung, breast, and renal solid tumor samples were obtained and shipped to Pierian Biosciences in Miltenyi Tissue Transport Buffer in Therapak controlled rate shippers maintained at 2-8° C. Upon receipt, tumors were cut into 2-3 mm pieces using sterile disposable scalpels and placed into tumor dissociation buffer (RPMI1640 plus pen/strep). The tumor pieces were then mechanically dissociated into a single cell suspension using enzymes from Miltenyi's human tumor dissociation kit and Miltenyi's gentleMACS™ Octo Dissociator with heaters. The single cell suspension was subsequently filtered through a 70 μM filter and counted. The cell suspension was then centrifuged and resuspended at the desired cell concentration in RPMI1640+10% FBS or 1×PBS+0.5% BSA.

After resuspending cells in the appropriate buffer, 80 μL were plated into designated wells of a deep well 96-well plate (2 mL volume). Subsequently, cells were initially stained using the amine reactive dye Alexa750 for 15 min to distinguish live versus dead cells. Cells were then washed 2× in stain buffer (1×PBS+0.5% BSA) using a Biotek ELx450 deep well plate washer. Cells were then modulated for 3 h using the Leukocyte Activation Cocktail, with BD GolgiPlug™ from Becton Dickinson. The Leukocyte Activation Cocktail, with BD GolgiPlug™ is a ready-to-use polyclonal cell activation mixture containing the phorbol ester, PMA (Phorbol 12-Myristate 13-Acetate), a calcium ionophore (Ionomycin) and the protein transport inhibitor BD GolgiPlug™ (Brefeldin A). After 3 h, cells were subsequently fixed and permeabilized utilizing the FOXP3/Transcription Staining Buffer Kit from eBioscience according to the manufacturers recommendations for processing and staining cells in a deep-well plate. Cells were then washed 2× in stain buffer (1×PBS+0.5% BSA) using a Biotek ELx450 deep well plate washer. Cells were then stained with fluorescently labeled antibody cocktails to detect CD3+, CD4+, CD8+ and Tregs (CD4+CD25^{hi}FoxP3+). Stained cells were incubated for 1 h at ambient temperature in the dark. Cells were then washed 2× in stain buffer (1×PBS+0.5% BSA) using a Biotek ELx450 deep well plate washer prior to fixation in PFA at a final concentration of 1%.

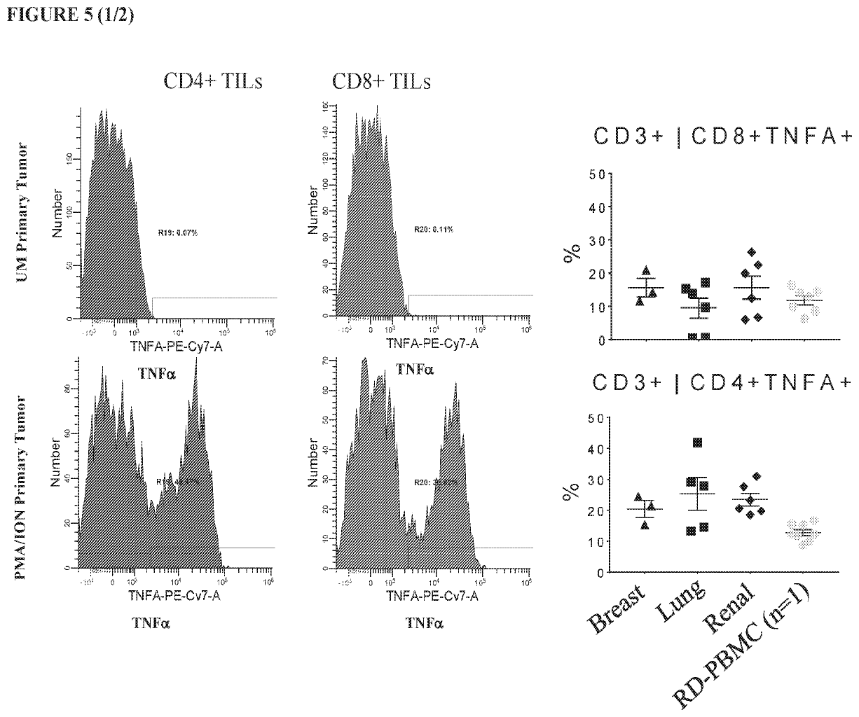

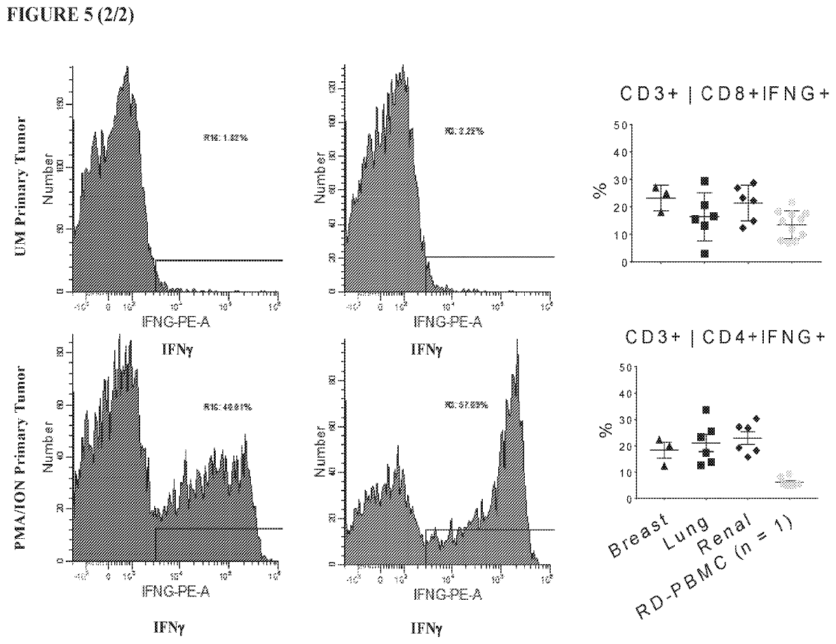

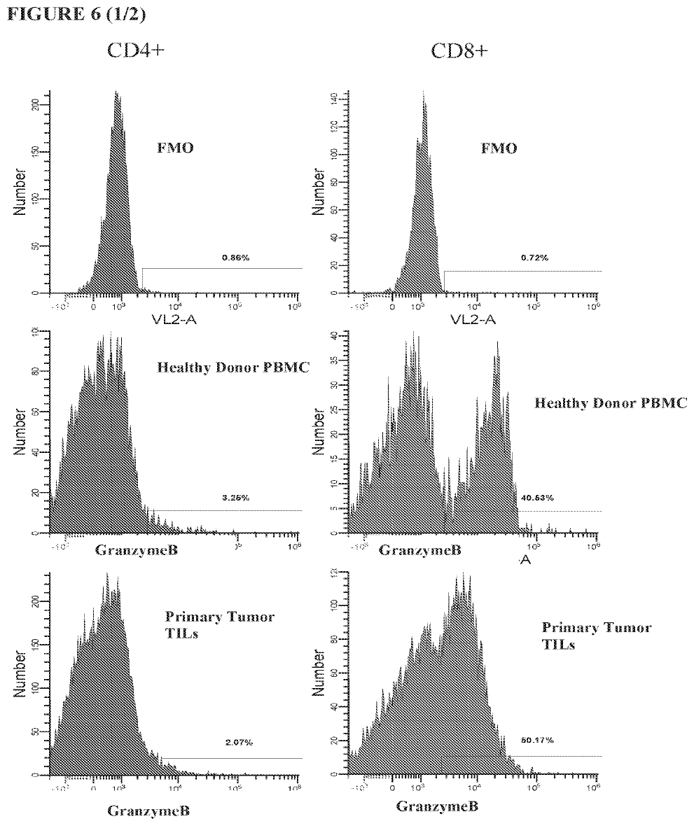

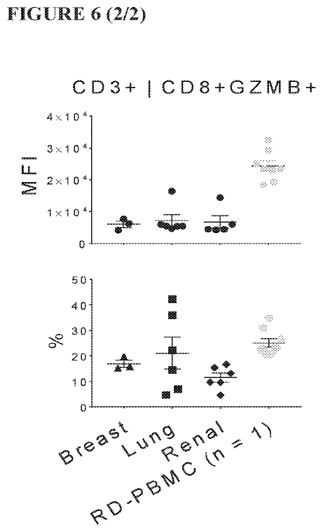

Stained cells were acquired directly from the 96-well deep well plate using a Thermo-Fisher Attune NxT 16-color flow cytometer. Prior to acquisition, all required information such as sample ID, antibody staining panels, and pre-determined and fixed flow cytometer instrument settings and acquisition parameters were incorporated into a 96 well plate layout using Ryvett software from Qognit, Inc. Post acquisition, FCS files were automatically analyzed and gated in Ryvett software utilizing pre-determined gating routines. Following manual review of auto-gating, raw data and calculated metrics were exported to a CSV file utilizing pre-defined criteria and data extraction routines. The results are shown in FIGS. 5-6. FIG. 5 shows exemplary flow cytometry plots demonstrating the detection of basal and induced IFNγ and TNFα in CD4+ and CD8+ T cells and the associated dot plots showing express of IFNγ and TNFα (expressed as percent positive). FIG. 6 shows exemplary flow cytometry plots demonstrating the detection of Granzyme B expression in CD4+ and CD8+ T cells from breast, lung, and renal tumors.

IFNγ and TNFα are cytokines indicative of an “inflamed” tumor with greater immune response potential with elevated levels of IFNγ and TNFα at initial immunotherapy treatment and subsequent follow up being associated with initial and durable responses, respectively. Granzyme B is an effector molecule found in CD8+ cells and is part of the Granzyme B-Perforin complex that CD8+ cells utilize to kill target tumor cells. Decreased levels of Granzyme B are associated with reduced CD8+ cytotoxic potential and an indicator of T cell exhaustion. Determining the percentage of CD8+ cells expressing Granzyme B in the tumor and the quantitative level of expression of Granzyme B is useful in predicting response to immunotherapy and developing a treatment strategy for the patient.

### Example 3—Assessing Immune Checkpoint and Exhaustion Markers and Their Cognate Ligands from Solid Tumors

In this Example, the expression levels of immune checkpoint/exhaustion markers and their cognate ligands on TILs and tumor epithelial cells were measured. Immunotherapy treatment currently depends on a single IHC measurement of the IMR ligand PD-L1 expression on tumors cells as treatment directing, however this does not sufficiently identify IMR and IMR-L expression on any other cellular component within the tumor microenvironment. A wholistic approach as described herein can improve patient stratification and treatment strategies.

In this example, breast, lung, and renal tumor samples were obtained and shipped to Pierian Biosciences in Miltenyi Tissue Transport Buffer in Therapak controlled rate shippers maintained at 2-8° C. Upon receipt, tumors were cut into 2-3 mm pieces using sterile disposable scalpels and placed into tumor dissociation buffer (RPMI1640 plus pen/strep). The tumor pieces were then mechanically dissociated into a single cell suspension using enzymes from Miltenyi's human tumor dissociation kit and Miltenyi's gentleMACS™ Octo Dissociator with heaters. The single cell suspension was subsequently filtered through a 70 μM filter and counted. The cell suspension was then centrifuged and resuspended at the desired cell concentration in RPMI1640+10% FBS or 1×PBS+0.5% BSA.

After resuspending cells in the appropriate buffer, 80 μL were plated into designated wells of a deep well 96-well plate (2 mL volume). Subsequently, cells were initially stained using the amine reactive dye Alexa750 for 15 min to distinguish live versus dead cells. Cells were then washed 2× in stain buffer (1×PBS+0.5% BSA) using a Biotek ELx450 deep well plate washer. Cells were then stained with fluorescently labeled antibody cocktails to detect CD326+, CD45+, CD3+, CD4+, CD8+, CD19+, CD56+, and CD14+ cells. Additional labeled antibodies were used to identify the following IMRs or IMR-Ls: CD73, CD112, CD155, CD172ab, CD274, CD279, CD366, TIGIT, TIM-3. Stained cells were incubated for 20 min at ambient temperature in the dark. Cells were then washed 2× in stain buffer (1×PBS+0.5% BSA) using a Biotek ELx450 deep well plate washer prior to fixation in PFA at a final concentration of 1%.

Stained cells were acquired directly from the 96-well deep well plate using a Thermo-Fisher Attune NxT 16-color flow cytometer. Prior to acquisition, all required information such as sample ID, antibody staining panels, and pre-determined and fixed flow cytometer instrument settings and acquisition parameters were incorporated into a 96 well plate layout using Ryvett software from Qognit, Inc. Post acquisition, FCS files were automatically analyzed and gated in Ryvett software utilizing pre-determined gating routines. Following manual review of auto-gating, raw data and calculated metrics were exported to a CSV file utilizing pre-defined criteria and data extraction routines.

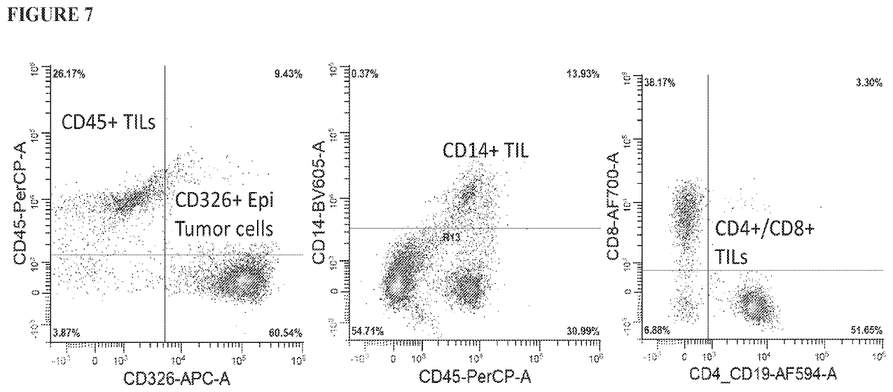

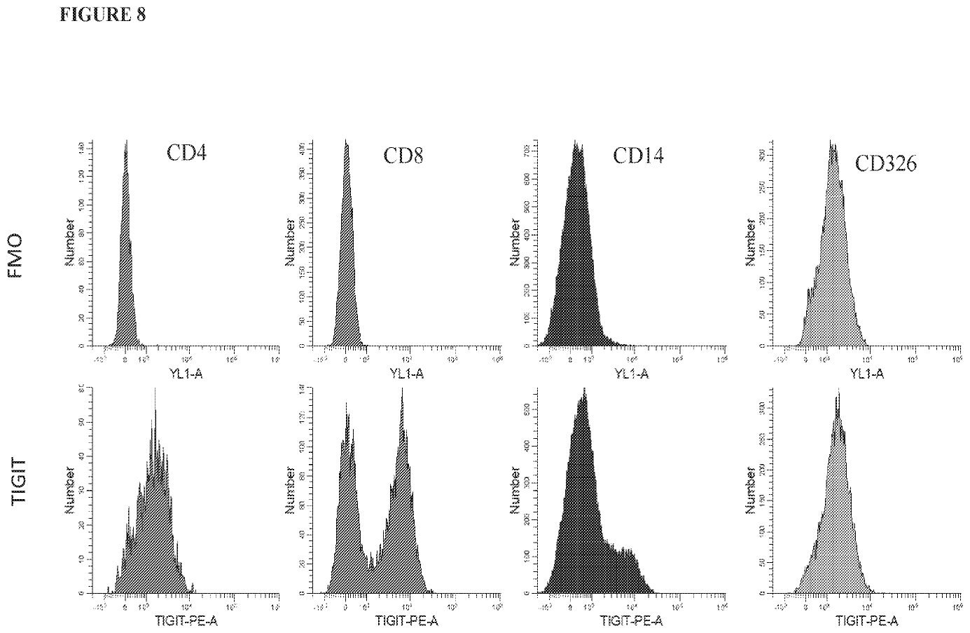

As shown in FIG. 7, the presence of CD45+ leukocytes, CD326+ epithelial tumor cells, CD14+ monocyte/macrophages and CD4+ and CD8+ T cells were detected within the tumor microenvironment. An example of inhibitory checkpoint receptor expression is shown in FIG. 8, where the inhibitory IMR TIGIT was detected on subsets of cells in the tumor microenvironment. Specifically, TIGIT is detected on CD4+, CD8+, and CD14+ leukocytes but not on CD326+ tumor cells.

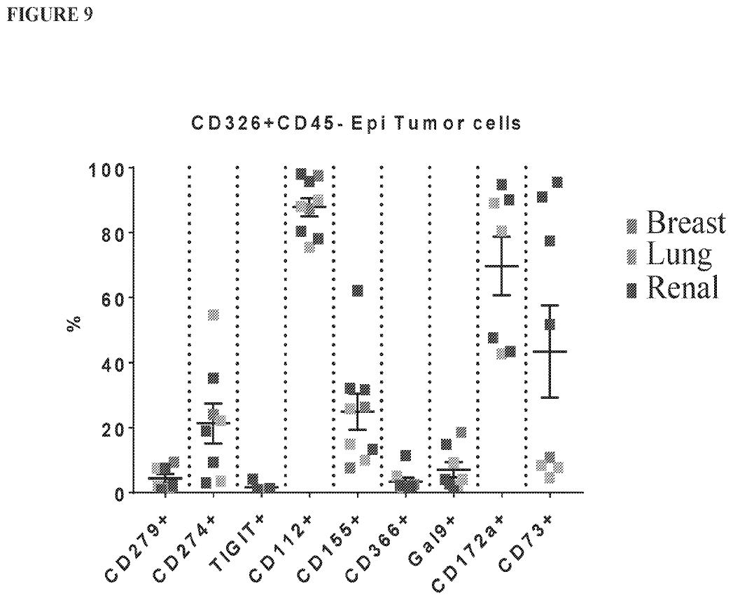

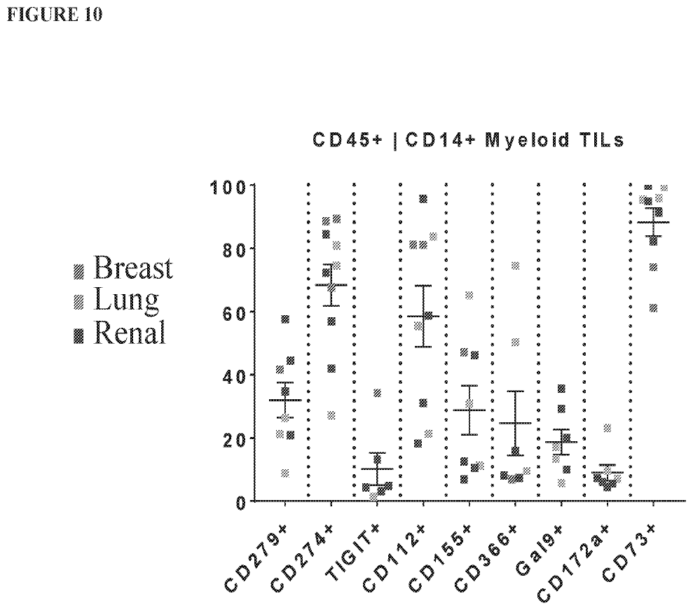

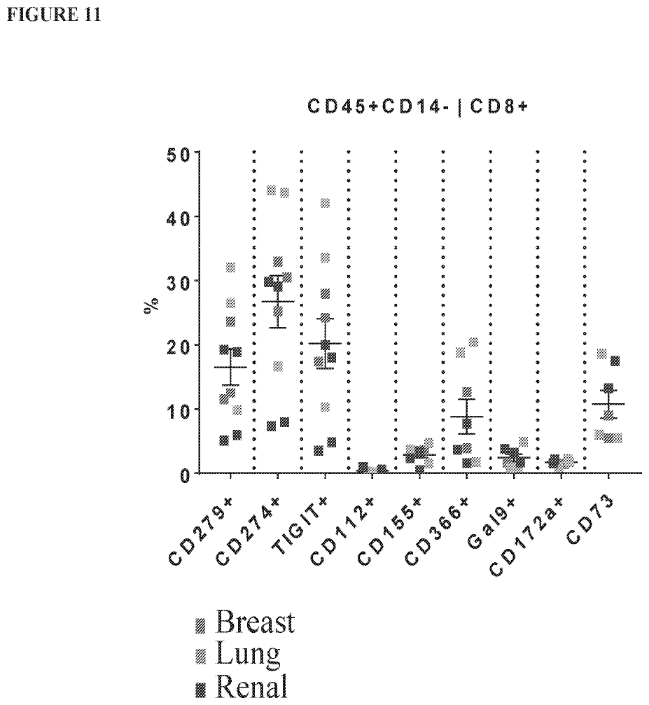

Levels of IMR/exhaustion markers and their ligands (CD279/CD274 [PD1/PD-L1], TIGIT/CD112-CD155, CD366 [TIM-3]/Galectin-9, CD172a [SIRPa]/CD47, CD73) were measured to profile the functional status of TILs. As shown in FIGS. 9-11, reliable separation of IMR/IMR-L positive TILs from negative TILs and IMR/IMR-L positive tumor cells from negative tumor cells was observed, with notable heterogeneity, across cell and tumor types (breast, lung, and renal). This included the elevated expression of several IMR-Ls on TILs and IMRs on epithelial and stromal cells, suggesting tumor- and TIL-intrinsic mechanisms modulating checkpoint interactions.

These results show that IMR/exhaustion markers and their ligands can be identified on specific cell types in the tumor microenvironment using the methods described herein. Understanding the composition of IMR/exhaustion markers and their ligands on specific cell types (e.g., immune cells) in the tumor microenvironment is important for determining the potential for a patient to respond to the prescribed immunotherapy.

### Example 4—Treating Patients with an Immunomodulator

A tumor sample is received from a patient and evaluated according to the methods described in Examples 1-3. A panel of IMR/exhaustion markers and their ligands (CD279/CD274 [PD1/PD-L1], TIGIT/CD112-CD155, CD366 [TIM-3]/Galectin-9, CD172a [SIRPa]/CD47, CD73) is measured to profile the functional status of TILs. The results show the presence of PD-1 (CD279) on T cells and PD-L1 (CD274) on one or more of dendritic cells, macrophages, and tumor cells. It is contemplated that if the subject is treated with an anti-PD-L1 or anti-PD-1 antibody the tumor may regress.

### Example 5—Treating Patients with a Combination of Immunomodulators

A tumor sample is received from a patient and evaluated according to the methods described in Examples 1-3. A panel of IMR/exhaustion markers and their ligands (CD279/CD274 [PD1/PD-L1], TIGIT/CD112-CD155, CD366 [TIM-3]/Galectin-9, CD172a [SIRPa]/CD47, CD73) is measured to profile the functional status of TILs. The results show the presence of PD-1 (CD279) on T cells and PD-L1 (CD274) on one or more of dendritic cells, macrophages, and tumor cells. The results further show the presence of immune checkpoint protein TIGIT on T cells and NK cells and the corresponding ligands CD112 and CD155 on one or more of dendritic cells, macrophages, and tumor cells. It is contemplated that if the subject is treated with both an anti-PD-L1 or anti-PD-1 antibody and an anti-TIGIT antibody, the tumor may regress.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of determining the composition of a solid tumor microenvironment, the method comprising the steps of:
(a) combining a single cell suspension of cells derived from a solid tumor with a plurality of labeling agents capable of binding a corresponding plurality of cell surface markers expressed on cancer cells and/or immune cells, wherein the cell surface markers comprise cell-type markers, immune modulatory receptors (IMRs) and IMR-ligands (IMR-Ls), and permitting the agents to simultaneously bind to cancer cells, immune cells or both cancer cells and immune cells present in the single cell suspension to produce labeled cells; and
(b) determining the presence and/or amount of the labeled cells by cytometry thereby to (i) determine the presence and/or amount of cancer cells, immune cells, or both cancer cells and immune cells present in the microenvironment of the solid tumor and (ii) determine whether the cancer cells, immune cells or both the cancer cells and immune cells express at least one of the IMRs and/or at least one of the IMR-Ls, thereby to determine the solid tumor microenvironment.

2. The method of claim 1, wherein the cytometry is selected from the group consisting of flow cytometry, mass cytometry, image cytometry, and a single cell technology (SCT).

3. The method of claim 1 or 2, wherein the plurality of labeling agents comprise at least one labeling agent selected from the group consisting of fluorophores, infrared labels, and heavy metal labels.

4. The method of any preceding claim, wherein the immune cells comprise lymphocytes (e.g., T cells (e.g., CD4+ T cells, CD8+ T cells, Tregs), B-cells, and natural killer cells), myeloid cells (e.g., dendritic cells, macrophages, and myeloid-derived suppressor cells), or a combination thereof.

5. The method of any preceding claim, wherein the cell surface markers further comprise cell activation markers.

6. The method of claim 5, wherein the cell-type markers comprise markers expressed on lymphocytes (e.g., T cells (e.g., CD4+ T cells, CD8+ T cells, Tregs), B-cells, and natural killer cells), and/or myeloid cells (e.g., dendritic cells, macrophages, and myeloid-derived suppressor cells).

7. The method of any preceding claim, wherein the cell-type markers are cancer cell markers.

8. The method of claim 7, wherein the cancer cell markers comprise CD44, CD47, CD49f, CD271, CD326, cytokeratin (intracellular), E-cadherin, and/or vimentin.

9. The method of claim 5, wherein the cell activation markers comprise CD25, CD26, CD27, CD28, CD38, CD40, CD44, CD62L, CD69, CD80, CD86, CD95, CD95L, CD127, CCR7 (CD197), and/or functional markers, e.g., IFNγ, TNFα, and/or other cytokines and/or Granzyme B.

10. The method of any one of claims 1-9, wherein the IMR or IMR-L markers comprise PD-1 (CD279), PD-L1 (CD274), CTLA-4 (CD152), LAG3 (CD223), OX40 (CD134), TIM3 (CD366), GITR (CD357), 4-1BB (CD137), KIR (CD158B), 2B4 (CD244), ICOS (CD278), IDO, TIGIT, CD73, CD39, CD172a (SIRPa), B7H4 (B7S1), VISTA (B7-H5), CD355 (CRTAM), KLRG1, CD160 (BY55, NK1, NK28), CD30 (TNFRSF8), CD224 (GGT1), CD226, CD272 (BTLA), and/or CD115 (CSF-1R).

11. The method of any one of claims 1-10, further comprising the step of combining the cells with an immunomodulator.

12. The method of claim 11, further comprising determining the effect of the immunomodulator on the expression of at least a portion of the cellular markers expression on the cancer and/or immune cells.

13. A method of determining whether a subject with solid tumor is likely to respond to an immunomodulator, the method comprising the steps of:
(a) combining a single cell suspension of cells derived from a solid tumor with a plurality of labeling agents capable of binding a corresponding plurality of cell surface markers expressed on cancer cells and/or immune cells, wherein the cell surface markers comprise cell-type markers, immune modulatory receptors (IMRs) and IMR-ligands (IMR-Ls), and permitting the agents to simultaneously bind to cancer cells, immune cells, or both cancer cells an immune cells present in the single cell suspension to produce labeled cells;
(b) combining at least a portion of the single cell suspension of cells with an immunomodulator; and
(c) determining (i) the presence and/or amount of the labeled cells by cytometry thereby to determine the presence and/or amount of cancer cells, immune cells or both cancer cells and immune cells present in the solid tumor and whether cancer cells, immune cells or both the cancer cells and immune cells express at least one of the IMRs and/or at least one of the IMR-Ls and (ii) the effect of the immunomodulator on the cellular markers on or in the cancer cells, immune cells or both the cancer cells and immune cells, thereby to determine whether the subject is likely to respond to the immunomodulator.

14. The method of claim 13, wherein the immunomodulator is combined with the single cell suspension before, during or after step (a).

15. The method of claim 13 or claim 14, wherein the cytometry is selected from the group consisting of flow cytometry, mass cytometry, image cytometry, and a single cell technology (SCT).

16. The method of any one of claims 13-15, wherein the plurality of labeling agents comprises at least one labeling agent selected from the group consisting of fluorophores, infrared labels, and heavy metal labels.

17. The method of any one of claims 13-16, wherein the immune cells comprise lymphocytes (e.g., T cells (e.g., CD4+ T cells, CD8+ T cells, Tregs), B-cells, and natural killer cells), myeloid cells (e.g., dendritic cells, macrophages, and myeloid-derived suppressor cells), or a combination thereof.

18. The method of any one of claims 13-17, wherein the cell surface markers further comprise cell activation markers.

19. The method of any one of claims 13-18, wherein the cell-type markers comprise markers expressed on lymphocytes (e.g., T cells (e.g., CD4+ T cells, CD8+ T cells, Tregs), B-cells, and natural killer cells), and/or myeloid cells (e.g., dendritic cells, macrophages, and myeloid-derived suppressor cells).

20. The method of any one of claims 13-19, wherein the cell-type markers are cancer cell markers.

21. The method of claim 20, wherein the cancer cell markers comprise CD44, CD47, CD49f, CD271, CD326, cytokeratin (intracellular), E-cadherin, and/or vimentin.

22. The method of claim 18, wherein the cell activation markers comprise CD25, CD26, CD27, CD28, CD38, CD40, CD44, CD62L, CD69, CD80, CD86, CD95, CD95L, CD127, CCR7 (CD197), and/or functional markers such as IFNγ, TNFα, or other cytokines and/or Granzyme B.

23. The method of any one of claims 13-22, wherein the IMR or IMR-L markers comprise PD-1 (CD279), PD-L1 (CD274), CTLA-4 (CD152), LAG3 (CD223), OX40 (CD134), TIM3 (CD366), GITR (CD357), 4-1BB (CD137), KIR (CD158B), 2B4 (CD244), ICOS (CD278), IDO, TIGIT, CD73, CD39, CD172a (SIRPa), B7H4 (B7S1), VISTA (B7-H5), CD355 (CRTAM), KLRG1, CD160 (BY55, NK1, NK28), CD30 (TNFRSF8), CD224 (GGT1), CD226, CD272 (BTLA), and CD115 (CSF-1R).

24. A method of treating a solid tumor in a subject in need thereof, the method comprising administering an effective amount of an immunomodulator to the subject thereby to treat the solid tumor, wherein the immunomodulator is selected by using a method comprising the steps of:
(a) combining a single cell suspension of cells derived from the solid tumor with a plurality of binding agents capable of binding a corresponding plurality of cell surface markers expressed on cancer cells and/or immune cells, wherein the cell surface markers comprise cell-type markers, immune modulatory receptors (IMRs) and IMR-ligands (IMR-Ls), and permitting the agents to simultaneously bind to the cancer cells, immune cells or both cancer and immune cells present in the single cell suspension to produce labeled cells;
(b) combining at least a portion of the single cell suspension of cells with an immunomodulator; and
(c) determining (i) the presence and/or amount of the labeled cells by cytometry thereby to determine the presence and/or amount of cancer cells, immune cells, or both cancer cells and immune cells present in the solid tumor and whether the cancer cells, immune cells or both the cancer cells and immune cells express at least one of the IMRs and/or at least one of the IMR-Ls and (ii) the effect of the immunomodulator on the cellular markers on or in the cancer cells, immune cells or both the cancer cells and immune cells thereby to determine whether the subject is likely to respond to the immunomodulator.

25. The method of claim 24, wherein the immunomodulator is combined with the single cell suspension before, during or after step (a).

26. The method of claim 24 or claim 25, wherein the cytometry is selected from the group consisting of flow cytometry, mass cytometry, image cytometry, and a single cell technology (SCT).

27. The method of any one of claims 24-26, wherein the plurality of labeling agents comprises at least one labeling agent selected from the group consisting of fluorophores, infrared labels, and heavy metal labels.

28. The method of any one of claims 24-27, wherein the immune cells comprise lymphocytes (e.g., T cells (e.g., CD4+ T cells, CD8+ T cells, Tregs), B-cells, and natural killer cells), myeloid cells (e.g., dendritic cells, macrophages, and myeloid-derived suppressor cells), or a combination thereof.

29. The method of any one of claims 24-27, wherein the cell surface markers further comprise cell activation markers.

30. The method of any one of claims 24-29, wherein the cell-type markers comprise markers expressed on lymphocytes (e.g., T cells (e.g., CD4+ T cells, CD8+ T cells, Tregs), B-cells, and natural killer cells), and/or myeloid cells (e.g., dendritic cells, macrophages, and myeloid-derived suppressor cells).

31. The method of any one of claims 24-30, wherein the cell-type markers are cancer cell markers.

32. The method of claim 31, wherein the cancer cell markers comprise CD44, CD47, CD49f, CD271, CD326, cytokeratin (intracellular), E-cadherin, and/or vimentin.

33. The method of claim 29, wherein the activation markers comprise CD25, CD26, CD27, CD28, CD38, CD40, CD44, CD62L, CD69, CD80, CD86, CD95, CD95L, CD127, CCR7 (CD197), and/or functional markers such as IFNγ, TNFα, and/or other cytokines and/or Granzyme B.

34. The method of any one of claims 24-33, wherein the IMR or IMR-L markers comprise PD-1 (CD279), PD-L1 (CD274), CTLA-4 (CD152), LAG3 (CD223), OX40 (CD134), TIM3 (CD366), GITR (CD357), 4-1BB (CD137), KIR (CD158B), 2B4 (CD244), ICOS (CD278), IDO, TIGIT, CD73, CD39, CD172a (SIRPa), B7H4 (B7S1), VISTA (B7-H5), CD355 (CRTAM), KLRG1, CD160 (BY55, NK1, NK28), CD30 (TNFRSF8), CD224 (GGT1), CD226, CD272 (BTLA), and/or CD115 (CSF-1R).

35. The method of any one of claims 1-34, wherein the plurality of different labeled cells are detected at the same time during cytometry.

36. The method of any one of claims 1-35, wherein the plurality of different cell surface markers are detected at the same time during cytometry.

37. The method of claim 36, wherein at least 14 different cell surface markers are detected at the same time.

38. The method of any one of claims 1-37, wherein receptor-ligand interactions between the labeled cells can be detected and optionally quantified.

39. The method of claim 38, wherein the receptor-ligand interactions comprise interactions between a checkpoint inhibitor and its cognate ligand.

40. The method of claim 39, wherein the receptor-ligand interactions can be selected from the interactions between PD-1 and PD-L1, CTLA-4 and B7-1 and/or B7-2, TIM-3 and Gal9, GITR and GITRL, OX-40 and OX40L, CD-27 and CD70, 4-1BB and 4-1BBL, and/or CD-40L and CD40.

41. The method of any one of claims 1-40, wherein the presence and/or amount of the cell activation markers, IMR markers, IMR-L markers or a combination of the activation markers and the IMR and/or IMR-L markers expressed on the cancer cells and/or immune cells is determined.
